(19) United States
(12) Patent Application Publication
(10) Pub. No.: US 2004/0142862 A1
(43) Pub. Date: Jul. 22, 2004

(54) Methods and compositions for modulating agonist-induced downregulation of G protein-coupled receptors

(75) Inventors: Jennifer L. Whistler, El Cerrrito, CA (US); Mark von Zastrow, San Carlos, CA (US); Stephen R. Murray, New York, NY (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/622,373

(22) Filed: Jul. 18, 2003

Related U.S. Application Data
(60) Provisional application No. 60/397,048, filed on Jul. 19, 2002.

(51) Int. Cl. A61K 38/17

(52) U.S. Cl. 514/12

(57) ABSTRACT

The present invention provides polypeptides (termed “GASP”) that modulate post-endocytic sorting of a variety of G protein-coupled receptors, thereby modulating agonist-induced downregulation of such receptors. The invention also provides methods of modulating agonist-induced downregulation of G protein-coupled receptors, as well as GASP polypeptides and anti-GASP antibodies. In addition, the invention provides a delta opioid receptor (DOR) polypeptide containing the GASP binding domain. The invention encompasses related polynucleotides, vectors, host cells, production methods, and compositions. Moreover, the invention includes methods for prescreening or screening for test agents that modulate agonist-induced downregulation of G protein-coupled receptors.

### CROSS-REFERENCE TO RELATED APPLICATIONS

This application claims the benefit of U.S. Provisional Application No. 60/397,048, filed Jul. 19, 2002, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant nos. NIH DA10711 and NRSA DA05844. The Government may have certain rights in the invention.

### FIELD OF THE INVENTION

The present invention relates to generally to the area of agonist-induced downregulation of G protein-coupled receptors, such as opioid receptors. In particular, the invention relates to methods and compositions for modulating agonist-induced downregulation of G protein-coupled receptors and related screening methods.

### BACKGROUND OF THE INVENTION

Ligand-induced endocytosis plays a critical role in the physiological regulation of many G protein-coupled receptors (GPCRs). Endocytosis of certain GPCRs is mediated by a mechanism involving non-visual (beta-)arrestins, which act as adaptor proteins to promote the concentration of activated, phosphorylated receptors in clathrin-coated pits (reviewed in (1, 2)). Although this mechanism of regulated endocytosis is highly conserved, the functional consequences of GPCR endocytosis are diverse. In general, trafficking of internalized GPCRs via a rapid recycling pathway restores the complement of functional receptors in the plasma membrane and thereby mediates resensitization of receptor-mediated signal transduction (see references 1, 3, 4 in References section, below). In contrast, sorting of internalized GPCRs to lysosomes promotes proteolytic downregulation of receptors, leading to a prolonged attenuation of cellular signal transduction, termed “agonist-induced &ownregulation” (3, 5, 6). Furthermore, the post-endocytic sorting of the beta-2 adrenergic receptor (B2AR) between recycling and degradative membrane pathways can itself be regulated (7), suggesting the existence of an additional layer of control in the membrane trafficking of certain GPCRs. Despite its fundamental importance to physiological regulation, relatively little is known about the mechanism controlling the endocytic sorting of GPCRs to lysosomes.

Mu (MOR) and delta (DOR) opioid receptors are structurally homologous GPCRs that mediate the actions of endogenously produced opioid neuropeptides and exogenously administered opiate drugs. Both receptors endocytose via clathrin-coated pits following agonist-induced activation, GRK-mediated phosphorylation and receptor interaction with cytoplasmic beta-arrestins (8, 9). Previous studies suggested that MOR and DOR differ in their physiological regulation and endocytic trafficking (6, 10, 12). However, the molecular mechanisms underlying these differences were previously unknown.

### SUMMARY OF THE INVENTION

The invention provides a method of inhibiting agonist-induced down-regulation of a G protein-coupled receptor. The method entails contacting cells containing the G protein-coupled receptor with an effective amount of an inhibitor, i.e., an amount sufficient to reduce agonist-induced down-regulation of the G protein-coupled receptor in the cells. The G protein-coupled receptor is one that specifically binds to a polypeptide having the amino acid sequence of GASP SEQ ID NO:2 (GASP1) or GASP SEQ ID NO:6 (GASP2). The inhibitor reduces specific binding of the G protein-coupled receptor to said polypeptide. In a particular embodiment, the inhibitor includes a polypeptide that reduces agonist-induced down-regulation of the G protein-coupled receptor and, more specifically, includes an amino acid sequence that has at least about 70% identity to GASP SEQ ID NO:2 (GASP1) or GASP SEQ ID NO:6 (GASP2) over a comparison window of at least 15 contiguous amino acids. In a variation of this embodiment, the amino acid sequence includes an amino acid subsequence of at least about 500 amino acids, of GASP SEQ ID NO:2 (GASP1) or GASP SEQ ID NO:6 (GASP2). In a preferred embodiment, the amino acid sequence defines a peptide that specifically binds to a G protein-coupled receptor. Preferred inhibitors according to the invention reduce agonist-induced down-regulation by at least about 20%, as determined by a radioligand binding assay. In one embodiment, the method additionally entails contacting the cells with an agonist of the G protein-coupled receptor in an amount sufficient to stimulate the G protein-coupled receptor.

Modulators of agonist-induced downregulation according to the invention can be contacted with the cells in vitro or in vivo. Polypeptides of the invention can be contacted with the cells by administering a composition comprising the polypeptide to the cells. Alternatively, a composition comprising a polynucleotide encoding the polypeptide can be administered to the cells, followed by expression of the encoded polypeptide.

In one embodiment, the G protein-coupled receptor is selected from the group comprising the delta opioid receptor, the kappa opioid receptor, the D2 dopamine receptor, the D4 dopamine receptor, the NK1 (substance P) receptor, the bradykinin B1 receptor, and US28. In a variation of this embodiment, the inhibitor of agonist-induced downregulation is contacted with the cells in vivo by administering a composition comprising the inhibitor to a subject in need of pain reduction.

The invention also provides a method of enhancing agonist-induced down-regulation of a G protein-coupled receptor. The method entails contacting cells containing the G protein-coupled receptor with an effective amount of a polypeptide that increases agonist-induced down-regulation of the G protein-coupled receptor. The G protein-coupled receptor is one that specifically binds to a polypeptide having the amino acid sequence of GASP SEQ ID NO:2 (GASP1) or GASP SEQ ID NO:6 (GASP2). The polypeptide that increases agonist-induced down-regulation includes an amino acid sequence that has at least about 70% identity to GASP SEQ ID NO:2 (GASP1) or GASP SEQ ID NO:6 (GASP2) over a comparison window of at least 15 contiguous amino acids. In a preferred embodiment, the amino acid sequence defines a polypeptide or peptide that specifically binds to a G protein-coupled receptor. Preferred polypeptides or peptides of this type increase agonist-induced down-regulation by at least about 20%, as determined by a radioligand binding assay. In one embodiment, the method additionally entails contacting the cells with an agonist of the G protein-coupled receptor in an amount sufficient to stimulate the G protein-coupled receptor.

Other aspects of the invention include compositions, such as GASP and DOR polypeptides. A GASP polypeptide includes an amino acid sequence, wherein the amino acid sequence has at least about 70% identity to GASP SEQ ID NO:2 (GASP1) or GASP SEQ ID NO:6 (GASP2) over a comparison window of at least 15 contiguous amino acids. In one embodiment, the amino acid sequence includes an amino acid subsequence of at least about 500 amino acids, of GASP SEQ ID NO:2 (GASP1) or GASP SEQ ID NO:6 (GASP2). In a preferred embodiment, the amino acid sequence defines a polypeptide peptide that specifically binds to a G protein-coupled receptor. In a variation of this embodiment, the amino acid sequence defines a peptide that reduces reduces agonist-induced down-regulation of the G protein-coupled receptor, preferably by at least about 20%, as determined by a radioligand binding assay.

A DOR polypeptide of the invention includes an amino acid sequence, wherein the amino acid sequence has at least about 70% identity to delta opioid receptor SEQ ID NO:3 over a comparison window of at least 15 contiguous amino acids. This amino acid sequence defines a peptide that specifically binds GASP SEQ ID NO:2 (GASP1) or GASP SEQ ID NO:6 (GASP2). The DOR polypeptide does not comprise more than about 50 contiguous amino acids of delta opioid receptor. In one embodiment, the amino acid sequence includes an amino acid sequence, or subsequence of at least about 30 amino acids, of delta opioid receptor SEQ ID NO:3.

Also within the scope of the invention are antibodies or antisera that specifically bind to the polypeptides of the invention, polynucleotides encoding the polypeptides of the invention, vectors including these polynucleotides, particularly expression vectors, and host cells containing such vectors. The invention also provides recombinant productions methods wherein a host cell containing an expression vector of the invention is cultured under conditions suitable for expression of the polypeptide, and the expressed polypeptide is recovered from the culture.

The invention additionally provides compositions including one or more of the polypeptides or polynucleotides of the invention in combination with a pharmaceutically acceptable carrier. In one embodiment, the composition additionally includes an agonist of the G protein-coupled receptor.

Furthermore, the invention includes prescreening and screening methods aimed at identifying agents that modulate agonist-induced downregulation of a G protein-coupled receptor of interest. One such prescreening method entails:

a) contacting a test agent with a polypeptide that specifically binds to the G protein-coupled receptor and modulates agonist-induced down-regulation of the G protein-coupled receptor, wherein the polypeptide comprises an amino acid sequence that has at least about 70% identity to GASP SEQ ID NO:2 (GASP1) or GASP SEQ ID NO:6 (GASP2) over a comparison window of at least 15 contiguous amino acids or with a polynucleotide encoding said polypeptide; and

b) detecting specific binding of the test agent to the polypeptide or polynucleotide.

Another such prescreening method entails:

a) contacting a test agent with a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2); and

b) detecting specific binding of the test agent to the receptor or receptor fragment.

A screening method of the invention entails:

a) contacting a test agent with a cell comprising:

(i) a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2); and

(ii) a GASP polypeptide comprising SEQ ID NO:2 or SEQ ID NO:6 or an allelic or species variant thereof;

b) determining the level of:

(i) GASP polypeptide;

(ii) GASP RNA; or

(iii) agonist-induced down-regulation of the G protein-coupled receptor.

### BRIEF DESCRIPTION OF THE DRAWINGS

FIGS. 1A-D: Post endocytic trafficking of opioid receptors.

1A. Agonist-induced downregulation of opioid receptors. Cells stably expressing MOR, DOR or D MOR (˜1-2 pmol/mg) were treated with agonist (10 μM DADLE, an opioid peptide having the sequence Tyr-D-Ala-Gly-Phe-D-Leu; SEQ ID NO:; Sigma, St. Louis, Mo.) for 3 hours or left untreated. Cells were then chilled on ice, washed extensively and total opioid radioligand binding sites were determined for each cell line (16). Both DOR and D MOR showed significant downregulation whereas MOR was not substantially downregulated under the same conditions (p<0.001). Error bars represent s.d. from a representative experiment (n=3 experiments), with each data point derived from triplicate determinations.

1B. Post endocytic sorting of MOR, DOR, D MOR, and R DOR was analyzed by biotin protection/degradation, which selectively follows the stability of endocytosed receptor protein (12, 17, 18). Endocytosed DOR and D MOR were extensively proteolyzed following 3 hours of agonist (Etorphine [NIDA], DADLE [Sigma], or DAMGO [Sigma]) treatment, whereas endocytosed MOR and R DOR were stable. Blots are representative of at least 2 independent experiments.

1C. Internalized DOR was colocalized with the lysosomal marker LAMP following 90 min. of agonist (5 μM DADLE) treatment (19) whereas MOR was not colocalized with LAMP following 90 min. of agonist (5 μM DAMGO; Sigma). Closed arrows show areas of the cells concentrated in LAMP. Open arrows show areas where MOR is distributed in areas without LAMP.

1D. Functional effects of opioid receptor membrane trafficking on signal transduction were assessed by membrane adenylyl cyclase assay (21) from stably transfected cells expressing MOR, DOR or D MOR. Rapid desensitization of all three receptors was observed following a brief (10 minute) exposure of cells to agonist (5 μM DAMGO for MOR and D MOR, 5 μM DADLE, for DOR). Significant differences were observed between cell lines following pretreatment with agonist for 3 hours followed by agonist washout and subsequent incubation with antagonist (10 μM naloxone; Sigma) for 30 minutes. MOR expressing cells recovered their ability to mediate agonist-dependent inhibition of adenylyl cyclase, consistent with resensitization of receptors mediated by recycling (11). In contrast neither DOR nor D MOR resensitized significantly under similar conditions, consistent with the postendocytic sorting to lysosomes and subsequent downregulation of these receptors (6, 12). Error bars represent s.d. from a representative experiment (n=2 experiments), with each data point derived from triplicate determinations.

FIGS. 2A-D: GASP1 and opioid receptor binding.

2A. Recombinant GASP1 produced by in vitro translation binds selectively to a GST fusion protein containing the cytoplasmic tail of DOR and with much reduced affinity to MOR (24).

2B. An antibody raised to a GASP1 peptide (27) recognizes an endogenous protein in HEK293 cells that co-electrophoreses with recombinantly expressed HA-tagged GASP1. Lysates were separated by SDS-PAGE, transferred to nitrocellulose and the blot was cut in half before immunoblotting (“IB”) with anti HA and anti-GASP1 antibodies.

2C. GASP1 selectively binds to DOR but not MOR in vivo (30). Cells stably expressing MOR, DOR (˜1-2 pmol/mg) or no receptor were lysed and receptors immunoprecipitated (“IP”). Precipitates were immunoblotted for GASP1 (upper blot) and receptor (middle blot). Lower blots shows lysate samples immunoblotted for GASP1. Less than 1% of endogenous GASP1 was immunoprecipitated with DOR.

2D. DOR binds GASP1 in vivo. HEK293 cells or HEK293 cells stably expressing DOR were lysed and GASP1 protein was immunoprecipitated. Precipitates were immunoblotted for receptor. 1% of the total DOR protein was recovered in the GASP1 immunoprecipitate; nevertheless, co-immunoprecipitations conducted in both directions were highly specific.

FIGS. 3A-E: cGASP1 binds to DOR and competes with full length GASP1 for DOR binding.

3A. Diagram showing the DOR binding hits from the two hybrid screen, the extent of cGASP1 and the epitope used to raise an antibody to GASP1.

3B. DOR binds a C-terminal fragment of GASP1, cGASP1, in vitro (24). Approximately 3-5% of input cGASP1 was recovered on washed GST-DOR.

3C. GFP-cGASP1 selectively binds to DOR but not MOR in vivo (30). Cells stably expressing GFP-cGASP1 alone or GFP-cGASP1 and DOR or MOR were lysed and receptors immunoprecipitated. Precipitates were immunoblotted specifically for cGASP1 using anti-GFP antibodies (upper blot) or receptor using anti-FLAG antibody (middle blot). Lower blot shows samples of cell lysate immunoblotted for GFP-cGASP1. Cells stably expressing both DOR and GFP were analyzed in parallel to ensure DOR was not coimmunoprecipitating with GFP.

3D. cGASP1 competes with GASP1 for DOR binding in vitro. Bacterially expressed MBP-DOR or MPB-lacZ was immobilized to amylose resin. In-vitro translated (IVT) full-length GASP1 bound selectively to the DOR cytoplasmic tail. Binding of IVT full-length GASP1 to MBP-DOR was competed in a concentration dependent manner by bacterially expressed and purified GST-cGASP1 protein (31).

3E. GFP-cGASP1 competes with endogenous GASP1 for binding to DOR in vivo (30). Receptor was immunoprecipitated from cells stably expressing DOR alone, DOR and GFP-cGASP1, or MOR. Precipitates were blotted for GASP1 using anti GASP1 antibodies to detect both GASP1 and cGASP1 (left blot) or anti-FLAG antibody to detect receptor (lower blot). Right blot shows lysate samples immunoblotted for GASP1, demonstrating that cGASP1 was overexpressed approximately 40 fold over endogenous GASP1. Immunoprecipitates were normalized for receptor (to assess the amount of GASP1 binding per receptor) not total protein, which explains the slight differences in GASP1 immunoreactivity on this blot. <1% of total endogenous GASP1 was coimmunoprecipitated with DOR. <1% of overexpressed cGASP1 coimmunoprecipitated with DOR.

FIGS. 4A-G: cGASP1 is a dominant inhibitor of DOR sorting to lysosomes.

4A. cGASP1 overexpression facilitates DOR recycling. Cells stably expressing FLAG-DOR were transiently transfected with HA-tagged-cGASP1 and cells were treated with agonist (Etorphine; NIDA) for 30 minutes (left panels) or agonist (Etorphine; NIDA) for 30 minutes followed by agonist washout and subsequent incubation with antagonist (Naloxone; Sigma) for 30 minutes (right panels). Endocytic trafficking of antibody-labeled DOR was examined by fluorescence microscopy (19). cGASP1 had no visible effect on agonist-induced endocytosis of receptors (left panels; note that numerous endocytic vesicles containing antibody-labeled DOR were observed in adjacent cells with and without overexpression of cGASP1). However, cGASP1 did affect receptor trafficking after agonist washout. In cells not overexpressing cGASP1 (right panels, closed arrows), antibody-labeled DOR remained predominantly in endocytic vesicles after agonist washout and subsequent incubation with the opiate antagonist naloxone for 30 minutes, consistent with efficient sorting of this receptor out of a recycling pathway (6). In contrast, in cells overexpressing cGASP1 (right panels, open arrows), increased recycling of internalized DOR was indicated by the appearance of antibody-labeled receptors in the plasma membrane after agonist washout.

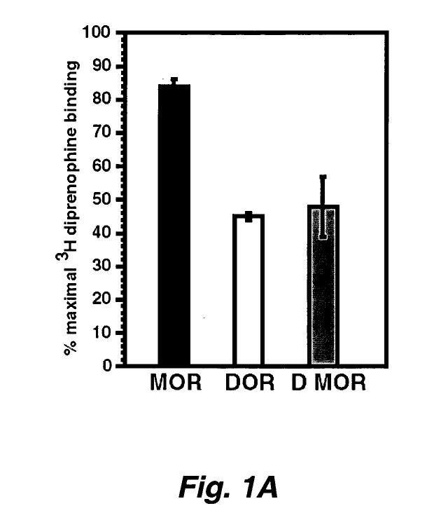

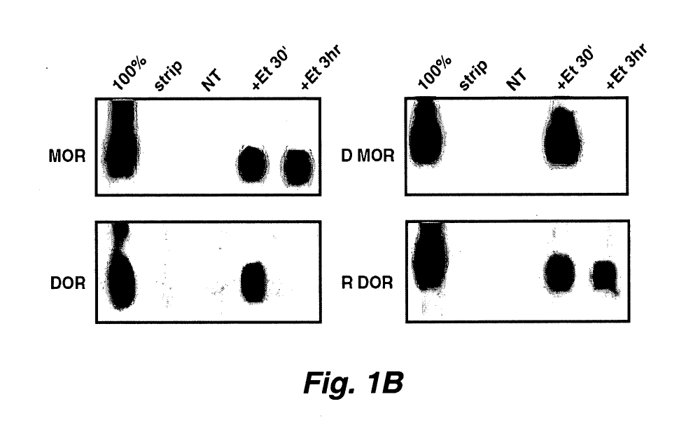

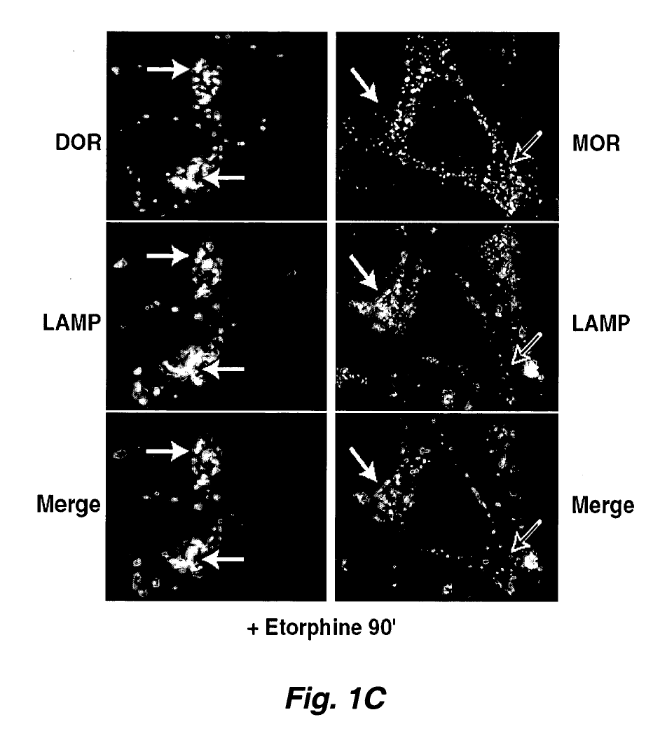

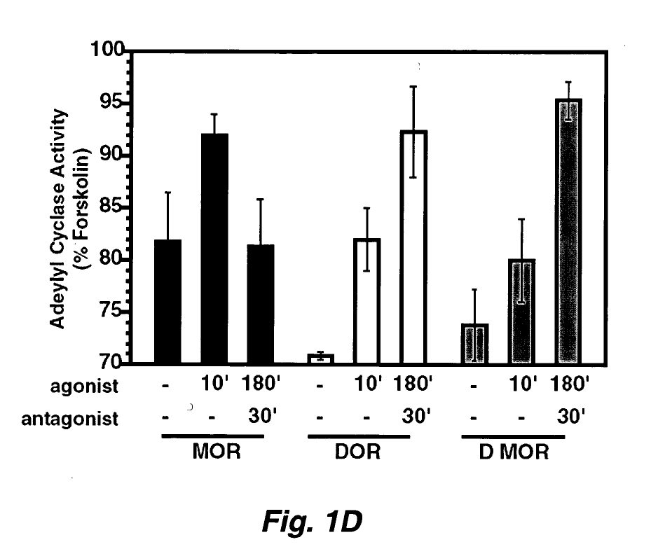

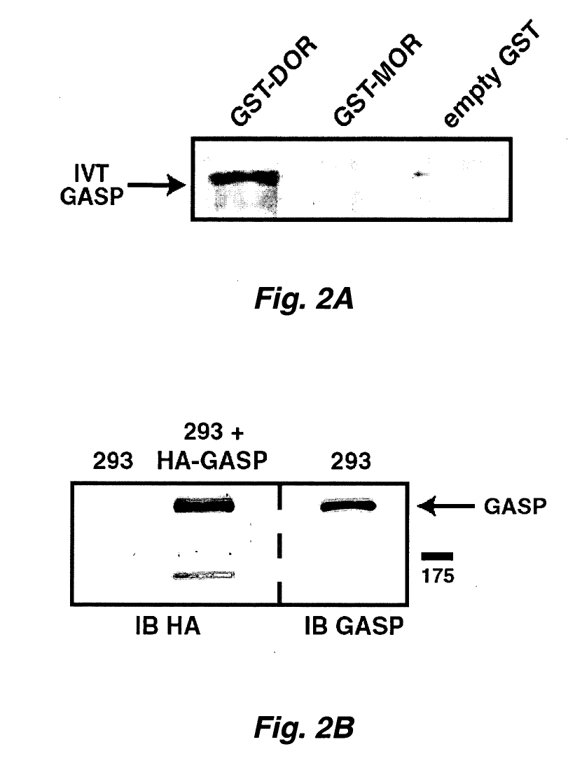

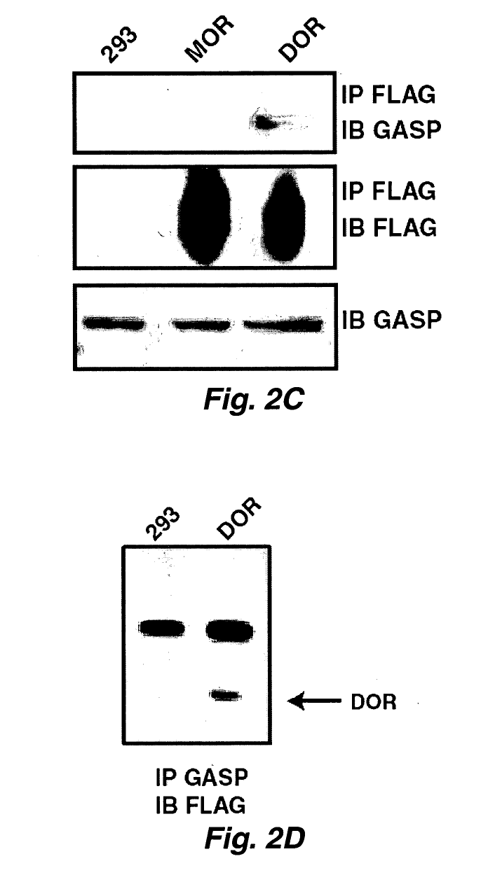

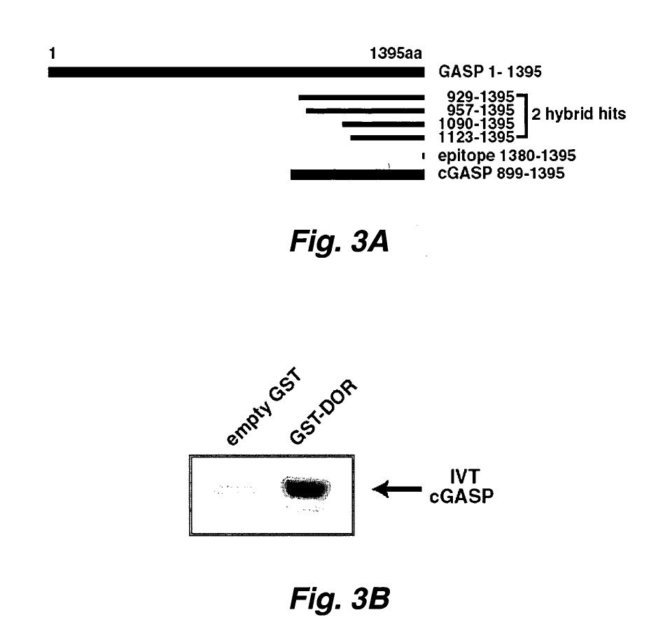

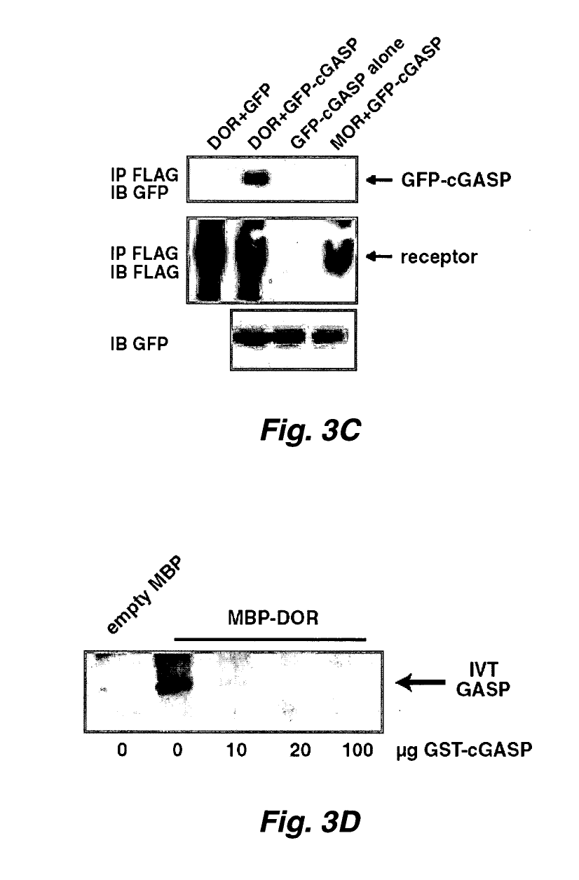

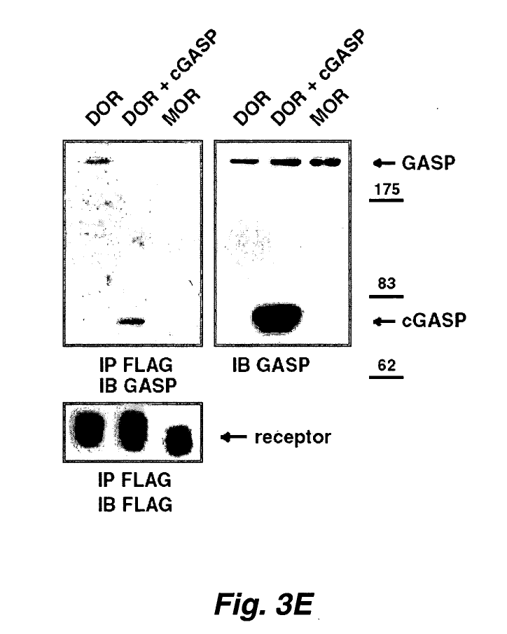

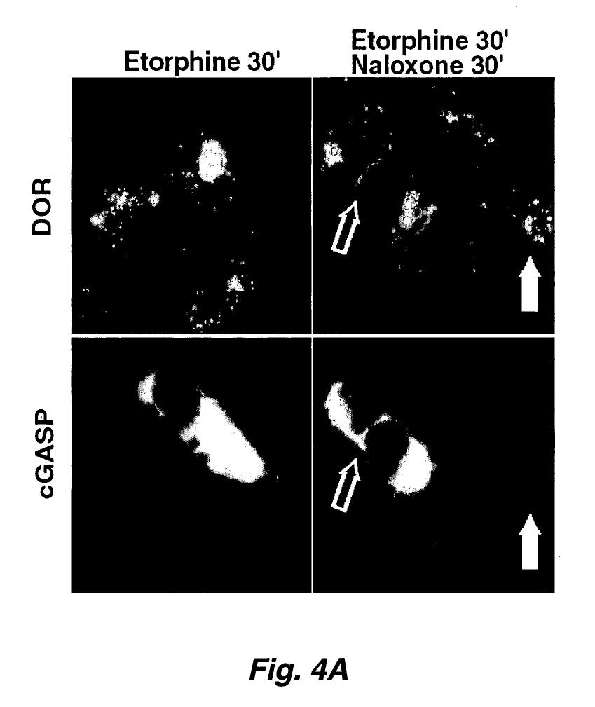

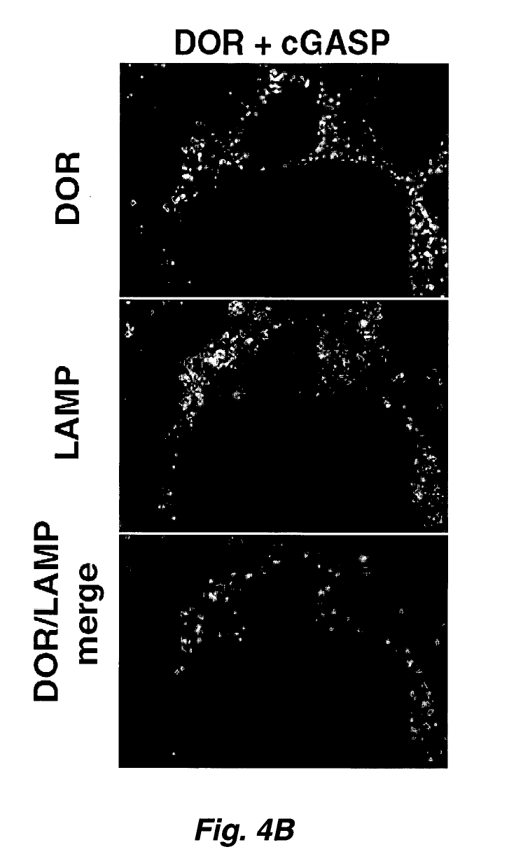

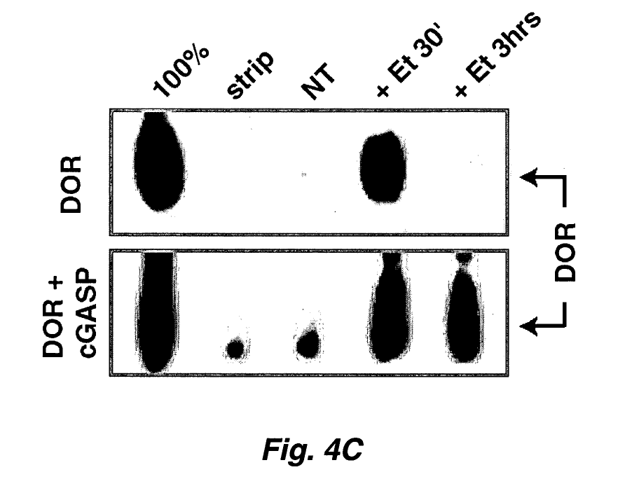

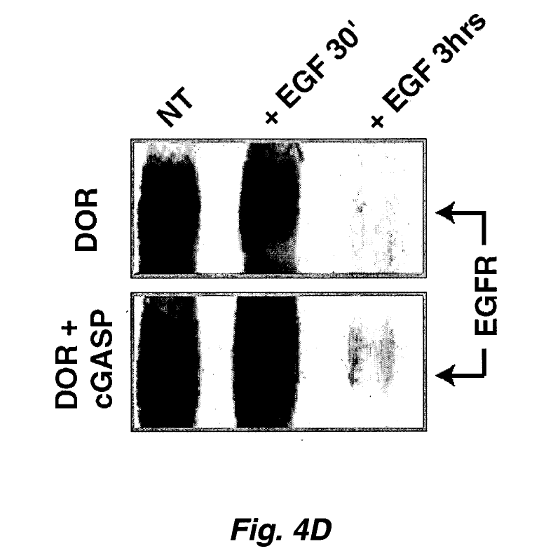

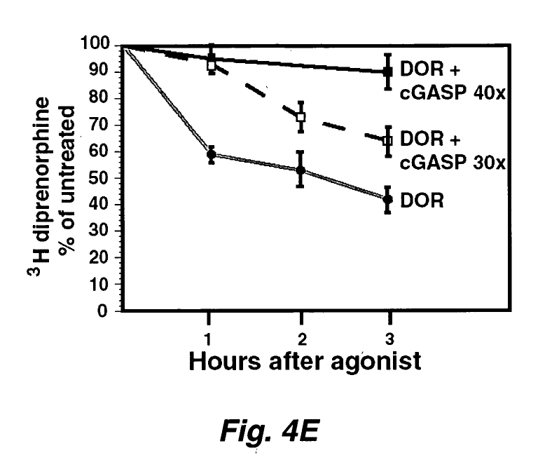

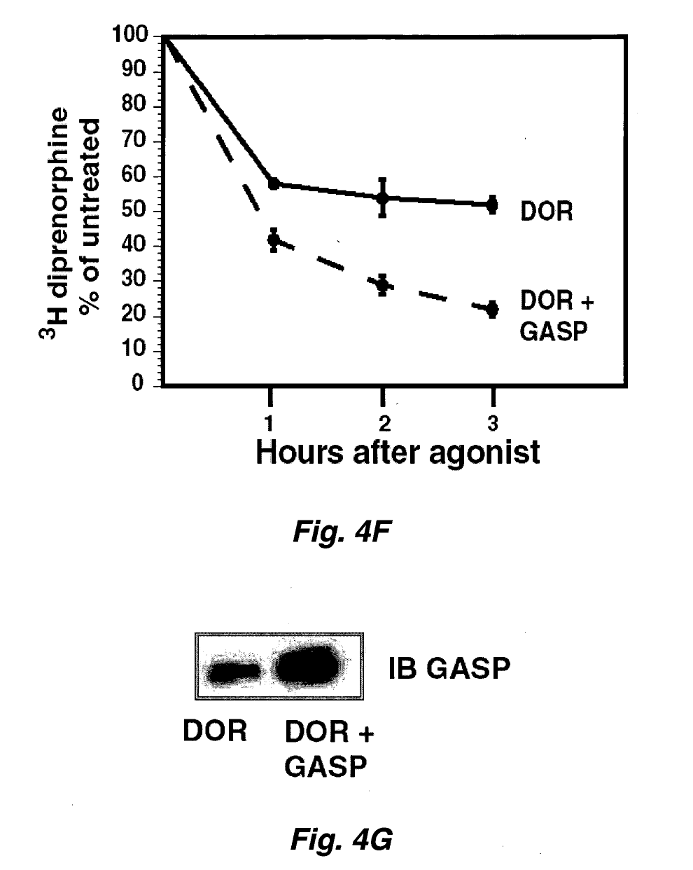

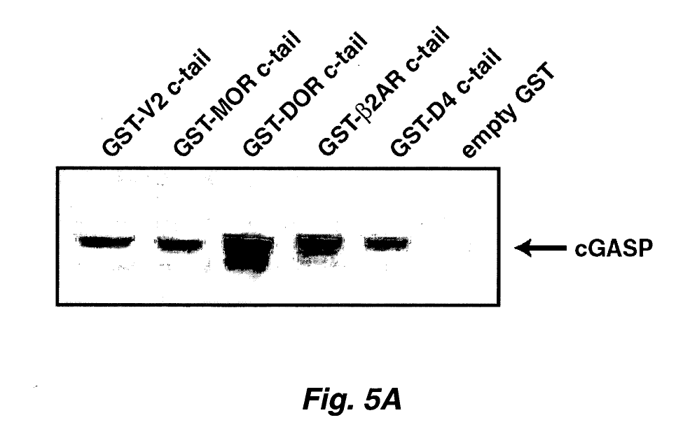

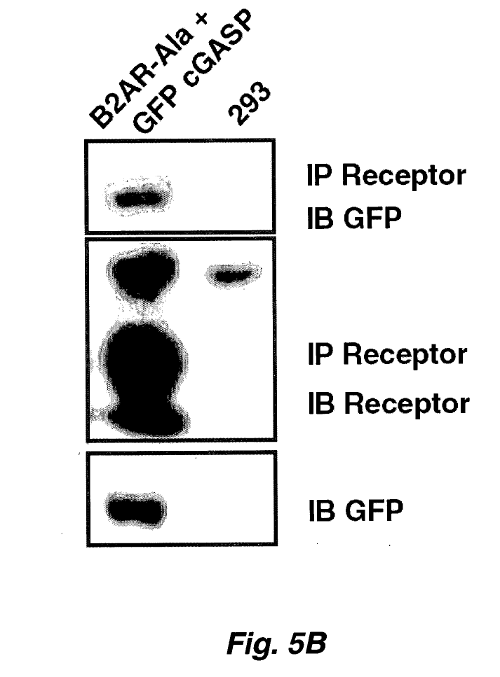

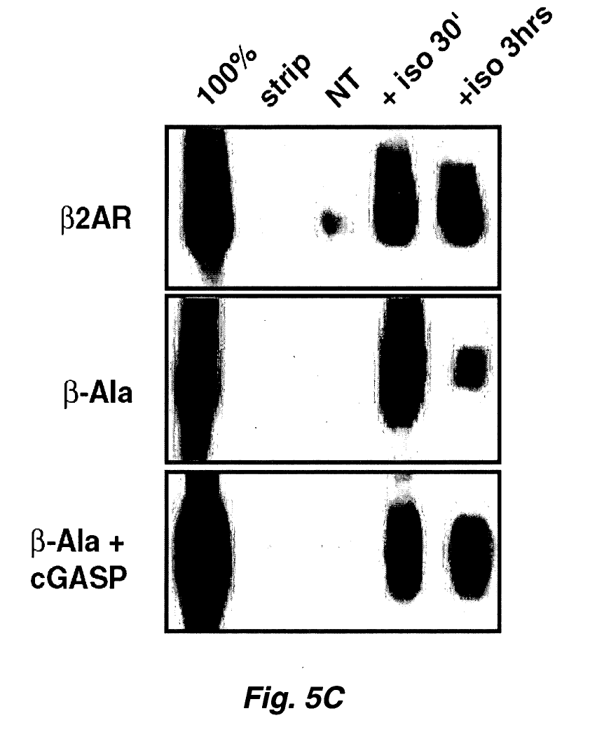

4B. cGASP1 blocks lysosomal targeting of DOR. Cells stably expressing both DOR and HA-cGASP1 (all cells express both) were treated with agonist (DADLE; Sigma) for 90 min. and stained for receptor and the lysosomal marker LAMP. There was little colocalization of receptor and LAMP (19), in contrast to the substantial colocalization observed under similar conditions in cells not overexpressing cGASP1 (FIG. 1C).

4C. cGASP1 inhibits post-endocytic proteolysis of DOR. The post-endocytic trafficking of DOR was assessed in cells stably expressing DOR alone or both DOR and GFP-cGASP1 using a biotin protection-degradation assay to selectively follow the stability of endocytosed receptors (17) (12, 18). Endocytosed DORs were significantly more stable in cells overexpressing cGASP1.

4D. Downregulation of the epidermal growth factor (EGF) receptor was unaffected by cGASP1 overexpression. DOR cells or DOR cells overexpressing GFP-cGASP1 were treated with EGF for 30 minutes or 3 hours or left untreated. EGF receptor was immunoprecipitated and analyzed by SDS-PAGE and immunoblot with anti EGFR antibodies (32). EGFR was as unstable in cells overexpressing cGASP1 as in cells that did not.

4E. cGASP1 inhibits agonist-induced downregulation of DOR measured by radioligand binding. Cells stably expressing DOR alone or DOR and cGASP1 were treated with agonist (5 μM DADLE) for 3 hours or left untreated. Cells were then washed extensively to remove residual agonist and total ligand binding sites present in cell lysates were determined (16). Agonist-induced downregulation of DOR was significantly inhibited in cells overexpressing cGASP140-fold over endogenous GASP1 (p<0.001) and less significantly in cells that overexpressed cGASP1 at only 30 fold over endogenous GASP1 (p<0.05). Error bars represent s.d. from a representative experiment (n=3 experiments), with each data point derived from triplicate determinations.

4F. Overexpression of full length GASP1 enhances DOR down-regulation. Cells stably expressing DOR and a GFP-tagged full length GASP1 (44), or expressing DOR alone were treated with agonist (DADLE; Sigma) for 1, 2 or 3 hours or left untreated. Cells were then washed extensively to remove residual agonist and total ligand binding sites present in cell lysates were determined (16). Agonist-induced downregulation of DOR was significantly enhanced both in rate and in extent in cells overexpressing GASP1. Error bars represent s.d. from a representative experiment (n=3 experiments), with each data point derived from triplicate determinations. g. Cells in (f) overexpress GASP1 approximately 4 fold. Subclones of cells stably transfected with GFP-GASP1 were selected for homogeneity in GFP fluorescence (44). Lysates were prepared and immunoblotted for GASP1 using the anti-GASP1 antibody.

FIGS. 5A-C: GASP1 interacts with a subset of other GPCRs.

5A. cGASP1 binds to the cytoplasmic tail of the beta 2 adrenergic receptor (B2AR) and somewhat to the D4 dopamine receptor, but not the V2 vasopressin receptor in vitro (24).

5B. GFP-cGASP1 binds in vivo to a mutant form of the B2AR, B2AR-Ala, that is sorted to lysosomes after endocytosis (7). Receptors from cells stably expressing B2AR-Ala and GFP-cGASP1 were immunoprecipitated and precipitates blotted for GFP (upper blot) or receptor (middle blot). Lower blot shows lysate samples immunoblotted for GFP-cGASP1 (30).

5C. cGASP1 inhibits post-endocytic proteolysis of B2AR-Ala. The post-endocytic trafficking of B2AR was assessed in cells stably expressing B2AR alone, B2AR-Ala alone or B2AR-Ala and c-GASP1 using the biotin protection-degradation assay to selectively follow the stability of endocytosed receptors (17) (12, 18). Endocytosed B2AR-Ala was significantly more stable in cells overexpressing cGASP1. (“iso” refers to cells treated with the B2AR agonist isoproterenol).

### DETAILED DESCRIPTION

The present invention provides a polypeptide that modulates post-endocytic sorting of a variety of G protein-coupled receptors, thereby modulating agonist-induced downregulation of such receptors. This polypeptide has been named “GASP” for “G protein-coupled receptor associated sorting protein. GASP binds specifically to the cytoplasmic tail of the delta opioid receptor (DOR) and other G protein-coupled receptors and promotes the sorting of bound receptors to lysosomes, leading to proteolytic downregulation. Disruption of this interaction promotes recycling of receptors to the plasma membrane, leading to functional resensitization of receptors.

The invention also provides methods of modulating agonist-induced downregulation of G protein-coupled receptors whose post-endocytic sorting is controlled by this mechanism, as well as GASP polypeptides and anti-GASP antibodies. The invention also provides DOR polypeptide containing the GASP binding domain. The invention encompasses related polynucleotides, vectors, host cells, production methods, and compositions. Moreover, the invention includes methods for prescreening or screening for test agents that modulate agonist-induced downregulation of G protein-coupled receptors that are downregulated by a GASP-dependent pathway.

Definitions

Terms used in the claims and specification are defined as set forth below unless otherwise specified.

The term “receptor” refers to a molecule or complex of molecules, typically (although not necessarily) a protein(s), that is specifically bound by one or more particular ligands. The receptor is said to be a receptor for such ligand(s). Ligand-receptor binding, in many instances, induces one or more biological responses.

A “G protein-coupled receptor” is a receptor that interacts with a “G protein” upon ligand-receptor binding. The term “G protein” refers to any heterotrimeric protein that binds GDP. Ligand-receptor binding stimulates a receptor-G protein interaction that results in the exchange GDP bound to the G protein for GTP. One or more G protein subunits then typically interact with one or more effectors or effector systems that mediate a biological response.

The following terms encompass polypeptides that are identified in Genbank by the following designations, as well as polypeptides that are at least about 70% identical to polypeptides identified in Genbank by these designations: mu opioid receptor, delta opioid receptor, kappa opioid receptor, D2 dopaminer receptor, D4 dopamine receptor, beta 2 adrenergic receptor, NK1(substance P) receptor, the bradykinin B1 receptor, and US28. In alternative embodiments, these terms encompass polypeptides identified in Genbank by these designations and polypeptides sharing at least bout 80, 90, 95, 96, 97, 98, or 99% identity.

An “agonist of a G protein-coupled receptor” is a ligand that specifically binds to and activates the G protein-coupled receptor, thereby stimulating at least one biological effect mediated by that receptor.

As used with reference to receptors, the term “down-regulation” refers to a reduction in the number of receptors on the surface of a cell. Down-regulation is conveniently measured using a ligand binding assay, such as a radioligand binding assay.

A “radioligand binding assay” is an assay in which a biological sample (e.g., cell, cell lysate, tissue, etc.) containing a receptor is contacted with a radioactively labeled ligand for the receptor under conditions suitable for specific binding between the receptor and ligand, unbound ligand is removed, and receptor binding is determined by detecting bound radioactivity. An exemplary radioligand binding assay described in Example 1 (see also 16).

The term “agonist-induced down-regulation” refers to a reduction in the number of cell surface receptors occurring after ligand binding and activation of the receptor.

An “inhibitor of agonist-induced down-regulation” is an agent that reduces, by any mechanism, the extent of agonist-induced down-regulation, as compared to that observed in the absence (or presence of a smaller amount) of the agent.

As used with respect to polypeptides or polynucleotides, the term “isolated” refers to a polypeptide or polynucleotide that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “polypeptide” and “protein” are used interchangeably herein to refer a polymer of amino acids, and unless otherwise limited, include a typical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “a typical” amino acids). For example, analogs or mimetics of phenylalanine or proline, which allow the same conformational restriction of the peptide compounds as natural Phe or Pro are included within the definition of “amino acid.”

Exemplary a typical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

As used with reference to a polypeptide, the term “full-length” refers to a polypeptide having the same length as the mature wild-type polypeptide.

The term “fragment” is used herein with reference to a polypeptide or a nucleic acid molecule to describe a portion of a larger molecule. Thus, a polypeptide fragment can lack an N-terminal portion of the larger molecule, a C-terminal portion, or both. Polypeptide fragments are also referred to herein as “peptides.” A fragment of a nucleic acid molecule can lack a 5′ portion of the larger molecule, a 3′ portion, or both. Nucleic acid fragments are also referred to herein as “oligonucleotides.” Oligonucleotides are relatively short nucleic acid molecules, generally shorter than 200 nucleotides, more particularly, shorter than 100 nucleotides, most particularly, shorter than 50 nucleotides. Typically, oligonucleotides are single-stranded DNA molecules.

A “subsequence” of an amino acid or nucleotide sequence is a portion of a larger sequence.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Residues in two or more polypeptides are said to “correspond”, if they are either homologous (i.e., occupying similar positions in either primary, secondary, or tertiary structure) or analogous (i.e., having the same or similar functional capacities). As is well known in the art, homologous residues can be determined by aligning the polypeptide sequences for maximum correspondence as described above.

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized.

The term “amino acid sequence variant” refers to a polypeptide having an amino acid sequence that differs from a wild-type amino acid sequence by the addition, deletion, or substitution of an amino acid.

The term “conservative amino acid substitution” is used herein to refer to the replacement of an amino acid with a functionally equivalent amino acid. Functionally equivalent amino acids are generally similar in size and/or character (e.g., charge or hydrophobicity) to the amino acids they replace. Amino acids of similar character can be grouped as follows:

(1) hydrophobic: His, Trp, Tyr, Phe, Met, Leu, Ile, Val, Ala;

(2) neutral hydrophobic: Cys, Ser, Thr;

(3) polar: Ser, Thr, Asn, Gln;

(4) acidic/negatively charged: Asp, Glu;

(5) charged: Asp, Glu, Arg, Lys, His;

(6) basic/positively charged: Arg, Lys, His;

(7) basic: Asn, Gln, His, Lys, Arg;

(8) residues that influence chain orientation: Gly, Pro; and

(9) aromatic: Trp, Tyr, Phe, His.

The following table shows exemplary and preferred conservative amino acid substitutions.

| | Exemplary | Preferred Conservative |
|---|---|---|
| Original Residue | Conservative Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Asn |
| Ile | Leu, Val, Met, Ala, Phe | Leu |
| Leu | Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

As used with reference to a polypeptide or polypeptide fragment, the term “derivative” includes amino acid sequence variants as well as any other molecule that differs from a wild-type amino acid sequence by the addition, deletion, or substitution of one or more chemical groups., “derivatives” retain at least one biological or immunological property of a wild-type polypeptide or polypeptide fragment, such as, for example, the biological property of specific binding to a receptor and the immunological property of specific binding to an antibody.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778).

The term “antiserum” refers to a polyclonal antibody typically raised by immunizing an animal with an immunogen and collecting serum containing polyclonal antibodies. The serum may be subjected to one or more purification steps, including affinity purification, to produce the antiserum.

The phrases “an effective amount” and “an amount sufficient to” refer to amounts of a biologically active agent to produce an intended biological activity.

A “signal sequence” is an amino acid sequence that directs the secretion of a polypeptide fused to the signal sequence. As used in recombinant expression, the polypeptide is secreted from a cell expressing the polypeptide into the culture medium for ease of purification.

An “epitope tag” is an amino acid sequence that defines an epitope for an antibody. Epitope tags can be engineered into polypeptides or peptides of interest to facilitate purification or detection. Exemplary epitope tags include the green fluorescent protein (GFP), hemagglutinin, and FLAG epitope tags, which are used in the studies described in Example 1.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

The term “vector” is used herein to describe a DNA construct containing a polynucleotide. Such a vector can be propagated stably or transiently in a host cell. The vector can, for example, be a plasmid, a viral vector, or simply a potential genomic insert. Once introduced into a suitable host, the vector may replicate and function independently of the host genome, or may, in some instances, integrate into the host genome.

As used herein, the term “operably linked” refers to a functional linkage between a control sequence (typically a promoter) and the linked sequence. For example, a promoter is operably linked to a sequence if the promoter can initiate transcription of the linked sequence.

“Expression vector” refers to a DNA construct containing a polynucleotide that is operably linked to a control sequence capable of effecting the expression of the polynucleotide in a suitable host. Exemplary control sequences include a promoter to effect transcription, an optional operator sequence to control transcription, a sequence encoding suitable mRNA ribosome binding sites, and sequences that control termination of transcription and translation.

The term “host cell” refers to a cell capable of maintaining a vector either transiently or stably. Host cells of the invention include, but are not limited to, bacterial cells, yeast cells, insect cells, plant cells and mammalian cells. Other host cells known in the art, or which become known, are also suitable for use in the invention.

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

A “test agent” is any agent that can be screened in the prescreening or screening assays of the invention. The test agent can be any suitable composition, including a small molecule, peptide, or polypeptide.

Methods of Modulating Agonist-Induced Downregulation of G Protein-Coupled Receptors

The invention provides methods of modulating agonist-induced downregulation of G protein-coupled receptors.

A. Method Of Inhibiting Agonist-Induced Downregulation Of G Protein-Coupled Receptors

1. In General

Inhibition of agonist-induced downregulation of receptors promotes the functional resensitization of receptors after agonist-induced activation and endocytosis. This effect can, for example, improve and/or prolong responsiveness to drugs or other agents whose effects are mediated by the particular receptor. Accordingly, the invention provides a method of inhibiting agonist-induced down-regulation of a G protein-coupled receptor entails contacting cells comprising the G protein-coupled receptor with an effective amount of an inhibitor. An effective amount is an amount sufficient to reduce agonist-induced down-regulation of the G protein-coupled receptor in the cells. In one embodiment, the method additionally entails contacting the cells with an agonist of the G protein-coupled receptor in an amount sufficient to stimulate the G protein-coupled receptor.

Any cell that has a suitable G protein-coupled receptor can be employed in the method. The cell is typically, although not necessarily, one that expresses the G protein-coupled receptor endogenously. The method generally employs animal cells, typically cells from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. Human cells can be employed.

The G protein-coupled receptor is one that specifically binds to a polypeptide having the amino acid sequence of GASP SEQ ID NO:2 (GASP1) or GASP SEQ ID NO:6 (GASP2). G protein-coupled receptors suitable for use in the method include the delta opioid receptor, the kappa opioid receptor, the D2 dopamine receptor, the D4 dopamine receptor, the beta 2 adrenergic receptor, the NK1 (substance P) receptor, the bradykinin B1 receptor, the US28, and the like. Additional G protein-coupled receptors having the requisite binding specificity can be determined using a binding assay, such as are described in Example 1. In general, G protein-coupled receptors that specifically bind to a polypeptide having the amino acid sequence of GASP SEQ ID NO:2 (GASP1) or GASP SEQ ID NO:6 (GASP2) with a higher affinity than MOR are preferred for use in the invention, and those having an affinity in approximately the same range (e.g., no more than about 5-fold less, preferably no more than about 2-fold less) as DOR are more preferred.

The inhibitor reduces specific binding of the G protein-coupled receptor to the GASP polypeptide, such that the binding observed in the presence of the inhibitor is less than that observed in the absence of inhibitor (or in the presence of a lower amount of inhibitor). Suitable inhibitors disrupt receptor-GASP binding, for example, by binding to the binding domain of one of the binding partners; binding near the domain and sterically hindering access to the domain; binding one of the binding partners and inducing a conformational change in the domain. In various embodiments, the inhibitor reduces agonist-induced down-regulation by at least about 10, 20, 30, 40, 50, 60, 70, 80, 90, and 95 percent, as determined by a radioligand binding assay.

In one embodiment, the inhibitor is a polypeptide that reduces agonist-induced down-regulation of the G protein-coupled receptor and includes an amino acid sequence that has at least about 70% identity to GASP SEQ ID NO:2 (GASP1) or GASP SEQ ID NO:6 (GASP2) over a comparison window of at least 15 contiguous amino acids. Percent identity can, for example, be determined by a sequence alignment performed using BLASTP with default parameters set to measure 70% identity. In variations of this embodiment, the percent identity is 80, 90, 95, 96, 97, 98, 99, or 100 percent. In a specific example of this embodiment, the amino acid sequence includes an amino acid subsequence of at least about 500 amino acids, of GASP SEQ ID NO:2 (GASP1) or GASP SEQ ID NO:6 (GASP2). The amino acid sequence can, for example, define a peptide that specifically binds to a G protein-coupled receptor. Preferably, the peptide reduces agonist-induced down-regulation of the G protein-coupled receptor, as shown in Example 1 for cGASP SEQ ID NO:2 (GASP1).

The cells are generally contacted with inhibitor under physiological conditions before or during a period of contact with an agonist for the G protein-coupled receptor. The duration of contact with the inhibitor can vary, depending on the particular application of the method. The duration of contact can range from minutes to days or longer. For research applications, the inhibitor is typically contacted with cells for, e.g., about 30 mins.; or about 1, about 3, about 6, or about 12 hours; or about 1, about 2, about 5, about 10, or about 15 days.

Contact of the inhibitor with cells can be achieved directly, i.e., by administering a composition containing the inhibitor to the cells, or indirectly, e.g., by administering a composition containing a polynucleotide encoding an inhibitor polypeptide to the cells. In the latter embodiment, this administration results in the introduction of the polynucleotide into one or more cells and the subsequent expression of the polypeptide in an amount sufficient to reduce agonist-induced down-regulation of the G protein-coupled receptor in the cells. In one embodiment, a composition containing a polynucleotide encoding the above-described peptide is administered to the cells.

This method can be carried out in vitro, i.e., in cells or tissues that are not part of an organism, or in vivo, in cells that are part of an organism. In one embodiment, cells are contacted in vitro in with an effective amount of an inhibitor (or a polynucleotide encoding the inhibitor).

Alternatively, cells can be contacted in vivo with a inhibitor by administering a composition containing the inhibitor (or a polynucleotide encoding the inhibitor) directly to a subject having, or at risk for, a condition that can be ameliorated by inhibiting agonist-induced downregulation of the G protein-coupled receptor. In one embodiment, for example, the subject is in need of pain reduction and the G protein-coupled receptor is one that modulates pain. Examples of G protein-coupled receptors the modulate pain and whose downregulation can be inhibited in this manner include the delta opioid receptor, the kappa opioid receptor, the D2 dopamine receptor, the D4 dopamine receptor, the NK1 (substance P) receptor, the bradykinin B1 receptor, and US28

2. Administration

For in vitro applications, cells are contacted with an inhibitor of the invention simply by adding the inhibitor or the polynucleotide encoding the inhibitor directly to the medium of cultured cells or directly to tissues.

Methods for in vivo administration do not differ from known methods for administering drugs or therapeutic polypeptides, peptides, or polynucleotides encoding them. Suitable routes of administration include, for example, topical, intravenous, intraperitoneal, intracerebral, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

3. Dose

The dose of inhibitor is sufficient to inhibit agonist-induced downregulation without significant toxicity. For in vivo applications, the dose of inhibitor depends, for example, upon the therapeutic objectives, the route of administration, and the condition of the subjected. Accordingly, it is necessary for the clinician to titer the dosage and modify the route of administration as required to obtain the optimal therapeutic effect. Generally, the clinician begins with a low dose and increases the dosage until the desired therapeutic effect is achieved. Starting doses for a given inhibitor can be extrapolated from in vitro data.

B. Method Of Enhancing Agonist-Induced Downregulation Of G Protein-Coupled Receptors

Enhancement of agonist-induced downregulation of receptors reduces the functional resensitization of receptors after agonist-induced activation and endocytosis. This effect can be exploited to reduce unwanted biological responses to agents whose effects are mediated by the particular receptor. For example, selective downregulation of particular receptor types can be used in the research setting to assign a particular biological response(s) to a particular receptor type, where an agonist binds to and activates several receptor types. Selective downregulation can also be employed therapeutically to confer greater specificity on a relatively non-specific drug. Thus, selective downregulation can be followed by administration of a drug that stimulates a G protein-coupled receptor that is susceptible to downregulation as described herein as well as a receptor that is not downregulated in this manner to bias the drug responses toward the those mediated by the latter receptor.

Accordingly, the invention provides a enhancing agonist-induced down-regulation of a G protein-coupled receptor that entails contacting cells comprising the G protein-coupled receptor with an effective amount of an enhancer of down-regulation. An effective amount is an amount sufficient to increase agonist-induced down-regulation of the G protein-coupled receptor in the cells. In one embodiment, the method additionally entails contacting the cells with an agonist of the G protein-coupled receptor in an amount sufficient to stimulate the G protein-coupled receptor.

Any cell that has a suitable G protein-coupled receptor can be employed in the method of enhancing agonist-induced downregulation, as described above for the method of inhibiting agonist-induced downregulation. Furthermore, both methods are applicable to the same G protein-coupled receptors.

The downregulation enhancer can increase agonist-induced down-regulation by at least about 10, 20, 30, 40, 50, 60, 70, 80, 90, and 95 percent, as determined by a radioligand binding assay. In particular embodiments, the downregulation enhancer is typically a polypeptide that increases agonist-induced down-regulation of the G protein-coupled receptor, such that the downregulation observed in the presence of the enhancer polypeptide is less than that observed in the absence of enhancer polypeptide (or in the presence of a lower amount of enhancer polypeptide). Exemplary enhancer polypeptides include an amino acid sequence that has at least about 70% identity to GASP SEQ ID NO:2 (GASP1) or GASP SEQ ID NO:6 (GASP2) over a comparison window of at least 15 contiguous amino acids. Percent identity can, for example, be determined by a sequence alignment performed using BLASTP with default parameters set to measure 70% identity. In variations of this embodiment, the percent identity is 80, 90, 95, 96, 97, 98, 99, or 100 percent. The amino acid sequence can, for example, define a peptide that specifically binds to a G protein-coupled receptor and, preferably, increases agonist-induced down-regulation of the G protein-coupled receptor.

The cells are generally contacted with downregulation enhancer under physiological conditions before or during a period of contact with an agonist for the G protein-coupled receptor. The duration of contact with the downregulation enhancer can vary, depending on the particular application of the method. The duration of contact can range from minutes to days or longer. For research applications, the downregulation enhancer is typically contacted with cells for, e.g., about 30 mins.; or about 1, about 3, about 6, or about 12 hours; or about 1, about 2, about 5, about 10, or about 15 days.

Contact of the downregulation enhancer with cells can be achieved directly, i.e., by administering a composition containing the downregulation enhancer to the cells, or indirectly, e.g., by administering a composition containing a polynucleotide encoding an enhancer polypeptide to the cells. In the latter embodiment, this administration results in the introduction of the polynucleotide into one or more cells and the subsequent expression of the enhancer polypeptide in an amount sufficient to increase agonist-induced down-regulation of the G protein-coupled receptor in the cells.

This method can be carried out in vitro, i.e., in cells or tissues that are not part of an organism, or in vivo, in cells that are part of an organism. In one embodiment, cells are contacted in vitro in with an effective amount of an downregulation enhancer (or a polynucleotide encoding the downregulation enhancer).

Alternatively, cells can be contacted in vivo with a downregulation enhancer by administering a composition containing the downregulation enhancer (or a polynucleotide encoding the downregulation enhancer) directly to a subject.

The considerations affecting administration and dose of downregulation enhancers do not differ from those set forth above with respect to the method of inhibiting agonist-induced downregulation.

GASP Polypeptides

A. Types of GASP Polypeptides

The invention also provides GASP polypeptides. A GASP polypeptide of the invention includes a GASP amino acid sequence, i.e., an amino acid sequence that has at least about 70% identity to GASP SEQ ID NO:2 (GASP1) or GASP SEQ ID NO:6 (GASP2) over a comparison window of at least 15 contiguous amino acids. GASP SEQ ID NO:2 (GASP1) is the amino acid sequence of a GASP polypeptide described in detail in Example 1. The nucleic acid and (single-letter code) amino acid sequences of these polypeptides are given below.

### GASP1 Nucleic Acid Sequence

SEQ ID NO: 1
atgactggggcagagattgagtctggtgcccaggtcaagcctgaaaagaagcctggggaagaggttgtaggtggggctgagata
gagaatgatgtccctctggtggtcagacccaaggttaggacccaggcccagataatgcctggggcaaggcccaagaataagtcca
aggttatgcctggagcaagcaccaaagttgagacaagtgcagtgggtggggcacgccctaagagtaaggccaaggcaatacctgt
ttcacgatttaaggaagaagcccagatgtgggctcagcccaggtttggtgctgaaagattgtctaagacagagagaaactcccagac
caatatcatagcctctccacttgtcagtactgattctgtcttggttgctaaaacaaagtacctgtctgaggatagagaactggttaataca
gacactgagagctttcctagaaggaaggcccattaccaagcaggattccagccttcttttaggtcaaaggaggagaccaatatgggg
tcctggtgctgtcctaggcctacatccaaacaagaagcctctcctaattctgatttcaaatgggtagacaaatctgtgagttccttgttct
ggagtggagatgaggtcactgcaaaatttcatcctgggaatagggtaaaagacagtaacagatccatgcacatggccaatcaagag
gctaataccatgtctaggtcccaaactaaccaggagctctatattgcatctagttctggttctgaggatgagtctgttaagacaccctgg
ttctgggccagagataaaaccaatacctggtctgggcccagggaagatcccaatagcaggtccaggtttaggtctaagaaagaagt
ctatgttgaatcaagttctggatctgagcatgaagaccatttggagtcctggtttggggctggaaaggagggcaaattcaggtccaaa
atgagagctgggaaggaggccaataacagggccaggcacagggccaagcgagaagcttgcattgatttcatgcctgggtctatag
atgtaattaaaaaagagtcctgtttctggcctgaagaaaatgctaataccttttcaaggcccatgatcaagaaagaggccagggccag
agcaatgacaaaggaagaggccaaaaccaaggcccgagccagggccaagcaagaagccaggtcagaggaggaagccctcatt
gggacctggttctgggctacagacgagtccagcatggcagatgaagccagcatagagtccagtctacaagtggaggatgagtcca
taattgggagttggttctggactgaagaagaggccagtatggggactggggctagcagtaaatccagaccaaggactgatgggga
gcgtattggtgattccttatttggggctagggaaaagaccagtatgaaaactggggctgaggccacctctgaatctatactagcagct
gatgatgaacaggtcattattggttcctggttctgggctggtgaagaggtcaaccaagaggctgaggaagagaccatttttgggtcgt
ggttctgggtcattgatgcggccagtgtggaatctggtgttggggtcagctgtgagtccaggacaaggtctgaggaagaagaggtc
attggtccctggttttggtctggagaacaagttgatatagaggctggaatcggagaagaggccaggccaggagctgaagaagaga
caatattcgggtcctggttttgggctgaaaaccagacctatatggattgtagggctgaaactagctgtgacaccatgcaaggggctga
ggaggaggagcccattattgggtcctggttttggaccagagtagaagcttgtgtggagggtgatgtcaacagcaagtctagcctgga
ggacaaggaagaggccatgataccatgttttggagccaaagaagaggtcagtatgaagcatgggactggtgtcagatgcagattta
tggcaggggctgaggagaccaataataagtcttgcttctgggcagaaaaagaaccctgtatgtatcctgccggtggaggaagttgg
aagtctaggccagaggaggaagaggacattgtcaattcgtggttctggtccagaaaatacacaaagccagaggccattatagggtc
ctggttatgggctacagaagagagtaatatagatgggactggagaaaaggccaagttactgactgaagaggagaccataatcaatt
cctggttctggaaagaagatgaagccatttcagaggctactgacagagaagagtccaggccagaagctgaggagggggacattgt
tggttcttggttctgggctggagaagaggacagactagagccagctgctgagactagagaagaagacaggctagcagctgagaaa
gaaggtattgttgggtcctggtttggggccagagaagagaccattagaagagaggctgggtcttgcagcaaatccagtcctaaagct
gaagaggaagaagtcattattgggtcctggttctgggaagaagaggccagtccggaggcagtggcaggagtcggctttgagtcaa
agcctgggactgaggaggaagaaatcactgttgggtcctggttctggcctgaagaagaagccagtatacaggctggatctcaggca
gtagaggaaatggagtcagagactgaagaggaaaccatttttgggtcctggttctgggatggaaaagaagtcagtgaagaagcag
gaccatgctgtgtatccaagccagaggatgatgaagagatgattgttgagtcctggttctggtctagagacaaagccattaaggaaac
tggaactgtggccacctgtgagtccaagccagaaaatgaggaaggggccattgttgggtcttggtttgaggctgaagatgaggtag
ataacaggactgacaatggaagcaactgtgggtccaggacattagctgatgaagatgaggccatagtggggtcctggttctgggca
ggagatgaggcccattttgaatcaaatcctagccccgtgttcagggccatttgcaggtccacgtgttcagttgaacaggagcctgatc
cttcacgcaggcctcagagttgggaggaggtcactgttcagttcaagcctggtccatggggtagggtcggcttcccatctataagcc
cctttagatttccgaaagaggcagcatctttattctgtgaaatgtttgggggcaaacccaggaacatggtacttagcccagaagggga
agatcaggaatctttgcttcagcctgatcagcctagtcctgagttcccatttcagtatgatccttcctacaggtcagtccaggaaattcga
gagcatcttagggccaaggagagtacagagcctgagagttcatcctgtaactgcatacaatgtgagctgaaaattggttctgaagagt
ttgaagaactccttttattaatggaaaaaattcgggatccttttattcatgaaatatctaaaatcgcaatgggtatgagaagtgcttctcaat
ttacccgagatttcattcgagattcaggtgttgtctcacttattgaaaccttgcttaattatccgtcctcccgagttagaacaagttttttgga
aaatatgattcgcatggccccaccttatccgaatctaaacataattcagacatacatatgtaaagtgtgtgaggaaacccttgcttatag
cgtggattccccggaacagctgtctggaataaggatgattagacatctcactactactactgactatcacacactggttgccaattatat
gtctgggtttctctccttattagctacaggcaatgccaaaacaaggtttcatgttttgaaaatgctactgaatttgtctgaaaatcttttcatg
acaaaagaactactcagtgctgaagcagtgtcagaatttataggcctctttaacagggaagagacaaatgacaatattcaaattgttctt
gcaatatttgagaatattggcaacaatatcaaaaaagaaacagtgttctctgatgatgatttcaatattgagccgcttatttctgcattcca
caaagttgagaaatttgctaaggaactgcaaggcaaaacagacaatcaaaatgaccctgaaggggaccaagaaaat

### GASP1 Amino Acid Sequence

| | |
|---|---|
| YWTFFVTCTMTGAEIESGAQVKPEKKPGEEVVGGAEIENDVPLVVRPKVRTQAQI | SEQ ID NO: 2 |
| MPGARPKNKSKVMPGASTKVETSAVGGARPKSKAKAIPVSRFKEEAQMWAQPRF | |
| GAERLSKTERNSQTNIIASPLVSTDSVLVAKTKYLSEDRELVNTDTESFPRRKAHYQ | |
| AGFQPSFRSKEETNMGSWCCPRPTSKQEASPNSDFKWVDKSVSSLFWSGDEVTAKF | |
| HPGNRVKDSNRSMHMANQEANTMSRSQTNQELYIASSSGSEDESVKTPWFWARD | |
| KTNTWSGPREDPNSRSRFRSKKEVYVESSSGSEHEDHLESWFGAGKEGKFRSKMR | |
| AGKEANNRARHRAKREACIDFMPGSIDVIKKESCFWPEENANTFSRPMIKKEARAR | |
| AMTKEEAKTKARARAKQEARSEEEALIGTWFWATDESSMADEASIESSLQVEDESII | |
| GSWFWTEEEASMGTGASSKSRPRTDGERIGDSLFGAREKTSMKTGAEATSESILAA | |
| DDEQVIIGSWFWAGEEVNQEAEEETIFGSWFWVIDAASVESGVGVSCESRTRSEEEE | |
| VIGPWFWSGEQVDIEAGIGEEARPGAEEETIFGSWFWAENQTYMDCRAETSCDTM | |
| QGAEEEEPIIGSWFWTRVEACVEGDVNSKSSLEDKEEAMIPCFGAKEEVSMKHGTG | |
| VRCRFMAGAEETNNKSCFWAEKEPCMYPAGGGSWKSRPEEEEDIVNSWFWSRKY | |
| TKPEAIIGSWLWATEESNIDGTGEKAKLLTEEETIINSWFWKEDEAISEATDREESRP | |
| EAEEGDIVGSWFWAGEEDRLEPAAETREEDRLAAEKEGIVGSWFGAREETIIRREAG | |
| SCSKSSPKAEEEEVIIGSWFWEEEASPEAVAGVGFESKPGTEEEEITVGSWFWPEEE | |
| ASIQAGSQAVEEMESETEEETIFGSWFWDGKEVSEEAGPCCVSKPEDDEEMIVESW | |
| FWSRDKAIKETGTVATCESKPENEEGAIVGSWFEAEDEVDNRTDNGSNCGSRTLAD | |
| EDEAIVGSWFWAGDEAHFESNPSPVFRAICRSTCSVEQEPDPSRRPQSWEEVTVQFK | |
| PGPWGRVGFPSISPFRFPKEAASLFCEMFGGKPRNMVLSPEGEDQESLLQPDQPSPE | |
| FPFQYDPSYRSVQEIREHLRAKESTEPESSSCNCIQCELKIGSEEFEELLLLMEKIRDP | |
| FIHEISKIAMGMRSASQFTRDFIRDSGVVSLIETLLNYPSSRVRTSFLENMIRMAPPYP | |
| NLNIIQTYICKVCEETLAYSVDSPEQLSGIRMIRHLTTTTDYHTLVANYMSGFLSLLA | |
| TGNAKTRFHVLKMLLNLSENLFMTKELLSAEAVSEFIGLFNREETNDNIQIVLAIFEN | |
| IGNNIKKETVFSDDDFNIEPLISAFHKVEKFAKELQGKTDNQNDPEGDQEN | |

### GASP2 Nucleic Acid Sequence

SEQ ID NO: 6
atgactggggcagagattgagcctagtgcccaggccaagcctgaaaagaaggctggggaagaggttatcgctgggcctgagaga
gagaatgatgtccctctggtggtcagacccaaggttaggacccaggcaactactggggcaaggcccaaaactgagaccaagtctg
tgcctgcggcaaggcccaaaactgaggcccaagcaatgtctggggcaaggcccaaaactgaggtccaagtaatgggtggtgcaa
gacccaaaacggaggctcaaggaatcacaggggccaggcccaaaaccgatgccagggcagtaggtggcgctcgttctaaaact
gatgccaaggcaatccctggagcaaggcccaaggatgaggcccaggcatgggcccagagtgaatttgggactgaagcagtgtca
caggcagaaggagtgtcccagactaatgccgttgcttggccactggccactgctgagtctggatcagttactaaatctaagggcctgt
ctatggatagagaactagtcaatgtggatgctgaaacctttcctggcacccagggtcagaaaggaatccagccctggtttggaccag
gggaggagactaatatggggtcttggtgctattccaggcccagggccagagaggaggcctctaatgagtctgggttctggtcagca
gatgagacctctacagcgtcttctttctggactggagaagagacaagtgtcagatcatggcccagggaagagtccaataccaggtcc
aggcacagggctaaacatcagactaatcccaggtccaggcccagatccaagcaagaagcctatgttgattcctggtctggatctga
ggatgaggccagcaacccattctccttctgggttggagaaaataccaataacttgttcaggcccagagtcagggaggaggcaaatat
caggtccaagctcaggacaaatagagaagattgttttgaatctgagtctgaagatgagttctataagcagtcctgggttttgcctggag
aagaggccaatagtagattcaggcacagagacaaagaagatcctaatactgccttgaaactcagggcccagaaagatgttgacagt
gatagggtcaaacaagaacccaggtttgaggaggaagtcattattgggtcctggttctgggcagaaaaagaggccagtttggaggg
tggagcttcagcaatctgtgaatctgagccaggaactgaggagggggccattggcggatccgcgtactgggctgaggaaaagtcc
agtttgggggctgtggccagagaagaggccaagccggagtctgaagaagaggccatatttgggtcctggttctgggacagagatg
aggcctgctttgacctaaatccctgtcctgtgtacaaggtcagtgataggttcagagatgcagctgaggagcttaatgcatcctccag
gccccaaacctgggacgaggtcactgttgaattcaaacctggtctttttcatggggttggcttccgatccacaagcccctttggaattcc
cgaagaggcttctgaaatgcttgaggcaaagcccaagaacctggaacttagcccagaaggagaagagcaggaatctttgcttcag
cctgatcagcctagtcctgagttcacatttcagtatgatccttcctaccggtcagtccgggaaattcgagagcatcttagggccaggga
gagtgcagagtctgagagttggtcctgcagctgcatacaatgtgagctgaaaattggttctgaagagtttgaagaattccttttattaatg
gacaaaattcgggatccttttattcatgaaatatctaaaattgcaatgggtatgagaagtgcttctcaatttacccgagatttcattcgaga
ttcaggtgttgtctcacttattgaaaccttgcttaattatccatcctctagagttaggacaagttttttggaaaatatgattcacatggctcca
ccttatccaaatctaaacatgattgagacattcatatgtcaagtgtgtgaggaaacccttgcacatagtgtggattcccttgagcagctg
actggaataaggatgcttagacacctcactatgactattgactatcacacactgattgccaactatatgtccgggtttctctccttattaac
cacagccaatgcgagaacgaagtttcacgttctgaaaatgctattgaatttgtctgaaaatcctgctgtggcaaaaaaactattcagtgc
caaagctctttcaatatttgtgggtctctttaacatagaagagacaaatgataatattcaaattgttattaaaatgtttcagaatatcagtaac
attataaaaagtggaaagatgtccttaattgatgatgatttcagtcttgagccgcttatttctgcatttcgtgaatttgaggagttagctaag
caactacaagcccaaatagacaaccaaaatgatcctgaggtgggacaacaaagttaa

### GASP2 Amino Acid Sequence

| | |
|---|---|
| MTGAEIEPSAQAKPEKKAGEEVIAGPERENDVPLVVRPKVRTQATTGARPKTETKS | SEQ ID NO: 6 |
| VPAARPKTEAQAMSGARPKTEVQVMGGARPKTEAQGITGARPKTDARAVGGARS | |
| KTDAKAIPGARPKDEAQAWAQSEFGTEAVSQAEGVSQTNAVAWPLATAESGSVTK | |
| SKGLSMDRELVNVDAETFPGTQGQKGIQPWFGPGEETNMGSWCYSRPRAREEASN | |
| ESGFWSADETSTASSFWTGEETSVRSWPREESNTRSRHRAKHQTNPRSRPRSKQEA | |
| YVDSWSGSEDEASNPFSFWVGENTNNLFRPRVREEANIRSKLRTNREDCFESESEDE | |
| FYKQSWVLPGEEANSRFRHRDKEDPNTALKLRAQKDVDSDRVKQEPRFEEEVIIGS | |
| WFWAEKLEASLEGGASAICESEPGTEEGAIGGSAYWAEEKSSLGAVAREEAKPESEE | |
| EAIFGSWFWDRDEACFDLNPCPVYKVSDRFRDAAEELNASSRPQTWDEVTVEFKP | |
| GLFHGVGFRSTSPFGIIPEEASEMLEAKPKNLELSPEGEEQESLLQPDQPSPEFTFQYD | |
| PSYRSVREIREHLRARESAESESWSCSCIQCELKIGSEEFEEFLLLMDKIRDPFIHEISK | |
| IAMGMRSASQFTRDFIRDSGVVSLIETLLNYPSSRVRTSFLENMIHMAPPYPNLNMIE | |
| TFICQVCEETLAHSVDSLEQLTGIRMLRHLTMTIDYHTLIANYMSGFLSLLTTANAR | |
| TKFHVLKMLLNLSENPAVAKKLFSAKALSIFVGLFNIEETNDNIQIVIKMFQNISNIIK | |
| SGKMSLIDDDFSLEPLISAFREFEELAKQLQAQIDNQNDPEVGQQS | |

Percent identity can, for example, be determined by a sequence alignment performed using BLASTP with default parameters set to measure 70% identity. In variations of this embodiment, the percent identity is 80, 90, 95, 96, 97, 98, 99, or 100 percent. The invention also encompasses polypeptides wherein the percent identities noted above are found over a comparison window of at least 20, 30, 40, 50, 60, 70, 80, 90, 100, 200, 300, 400, 500, 600, 700, 800, 900, or 1000 or more contiguous amino acids. In various embodiments, the amino acid sequence includes an amino acid sequence or subsequence of at least about 100, 200, 300, 400, 500, 600, 700, 800, 900, or 1000 or more amino acids, of GASP SEQ ID NO:2 (GASP1) or GASP SEQ ID NO:6 (GASP2). In one embodiment, that amino acid sequence is full-length human GASP, i.e., GASP SEQ ID NO:2 (GASP1) or GASP SEQ ID NO:6 (GASP2).

The GASP amino acid sequence can, for example, define a polypeptide or peptide that specifically binds to a G protein-coupled receptor. Exemplary G protein-coupled receptors that are bound by the GASP polypeptides of the invention include the delta opioid receptor, the kappa opioid receptor, the D2 dopamine receptor, the D4 dopamine receptor, the beta 2 adrenergic receptor, the NK1 (substance P) receptor, the bradykinin B1 receptor, the US28, and the like. Additional G protein-coupled receptors that are bound by GASP polypeptides can be determined using a binding assay, such as are described in Example 1. Generally, a GASP polypeptide of the invention specifically binds to a G protein-coupled receptor that is itself capable of specifically binding to wild-type human GASP1 or 2 (SEQ ID NO:2 or SEQ ID NO:6, respectively) with a higher affinity than MOR and, preferably, an affinity in approximately the same range (e.g., no more than about 5-fold less, preferably no more than about 2-fold less) as DOR.

In preferred embodiments, GASP polypeptides of the invention modulate agonist-induced downregulation of one or more G protein-coupled receptor(s) to which the GASP polypeptides specifically bind. The invention provides GASP polypeptides that reduce, as well as those that increase, agonist-induced down-regulation of the G protein-coupled receptor. The magnitude of these effects can be at least about 10, 20, 30, 40, 50, 60, 70, 80, 90, and 95 percent, as determined by a radioligand binding assay. In a specific example of this embodiment, a GASP polypeptide that inhibits downregulation includes cGASP SEQ ID NO:2 (GASP1) (see Example 1). By contrast, an exemplary GASP polypeptide that enhances downregulation includes full-length GASP SEQ ID NO:2 (GASP1). The corresponding GASP2 polypeptides function in the same manner.

The GASP amino acid sequence can be derived from any GASP-like polypeptide from any organism. GASP amino acid sequences useful in the invention are generally those derived from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. In particularly preferred embodiments, the GASP amino acid sequence is derived from a human polypeptide.

The GASP amino acid sequence can be a wild-type amino acid sequence or an amino acid sequence variant of the corresponding region of a wild-type polypeptide. Preferred GASP polypeptides generally include a wild-type GASP amino acid sequence or a GASP amino acid sequence containing conservative amino acid substitutions, as defined above.

In addition to the amino acid sequences described above, GASP polypeptides of the invention can include other amino acid sequences, including those from heterologous proteins. Accordingly, the invention encompasses fusion polypeptides in which the above-discussed amino acid sequence is fused, at either or both ends, to amino acid sequence(s) from one or more heterologous proteins. Examples of additional amino acid sequences often incorporated into proteins of interest include a signal sequence, which facilitates purification of the protein, and an epitope tag, which can be used for immunological detection or affinity purification.

Polypeptides of the invention can be otherwise modified to produce derivatives that retain the above-described functions, namely specific binding to G protein-coupled receptors and/or modulation of agonist-induced downregulation. In preferred embodiments, the modified polypeptides have an activity that is about 0.1 to about 0.01-fold that of the unmodified forms. In more preferred embodiments, the modified polypeptides have an activity that is about 0.1 to about 1-fold that of the unmodified polypeptides. In even more preferred embodiments, the modified polypeptides have an activity that is greater than the unmodified polypeptides.

Those of skill in the art recognize that a variety of techniques are available for constructing so-called “peptide mimetics” with the same or similar desired biological activity as the corresponding peptide compound, but with more favorable activity than the peptide with respect to, e.g., solubility, stability, and susceptibility to hydrolysis and proteolysis. See, for example, Morgan, et al., Ann. Rep. Med. Chem., 24:243-252 (1989). Accordingly, the GASP polypeptides of the invention include peptide mimetics that are, for example, modified at the N-terminal amino group, the C-terminal carboxyl group, and/or at one or more of the amido linkages in the peptide to a non-amido linkage.

B. Production of GASP Polypeptides

1. Synthetic Techniques

GASP polypeptides according to the invention can be synthesized using methods known in the art, such as for example exclusive solid phase synthesis, partial solid phase synthesis, fragment condensation, and classical solution synthesis. See, e.g., Merrifield, J. Am. Chem. Soc., 85:2149 (1963). Solid phase techniques are preferred. On solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories, Richmond, Calif., and the preparation of the hydroxymethyl resin is described by Bodonszky, et al., Chem. Ind. (London), 38:1597 (1966). The benzhydrylamine (BHA) resin has been described by Pietta and Marshall, Chem. Commn., 650 (1970) and is commercially available from Beckman Instruments, Inc., Palo Alto, Calif., in the hydrochloride form. Automated peptide synthesizers are commercially available, as are services that make peptides to order.

Thus, the polypeptides of the invention can be prepared by coupling an alpha-amino protected amino acid to the chloromethylated resin with the aid of, for example, cesium bicarbonate catalyst, according to the method described by Gisin, Helv. Chim. Acta., 56:1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, acetyl), aromatic urethane type protecting groups (e.g. benzyloxycarboyl (Cbz) and substituted Cbz), aliphatic urethane protecting groups (e.g., t-butyloxycarbonyl (Boc), isopropyloxycarbonyl, cyclohexyloxycarbonyl), and alkyl type protecting groups (e.g., benzyl, triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride, dimethyl formamide (DMF) mixtures.

After the desired amino acid sequence has been completed, the desired peptide is decoupled from the resin support by treatment with a reagent such as trifluoroacetic acid or hydrogen fluoride (HF), which not only cleaves the peptide from the resin, but also cleaves all remaining side chain protecting groups. When the chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well known in the art and further described by John Morrow Stewart and Janis Dillaha Young, Solid Phase Peptide Syntheses (2nd Ed., Pierce Chemical Company, 1984).

2. Recombinant Techniques

GASP polypeptides can also produced using recombinant techniques. Precursor GASP genes or gene sequences can be cloned, for instance, based on homology to the GASP polypeptides described herein. With a precursor GASP gene in hand, a nucleic acid molecule encoding a desired GASP polypeptide can be generated by any of a variety of mutagenesis techniques. See, e.g., Sambrook, J., Fritsch, E. F., and Maniatis, T. (1989) in Molecular Cloning: A Laboratory Manual, Second Edition, Cold Spring Harbor, N.Y. Examples include site-specific mutagenesis (Kunkel et al., (1991) Methods Enzymol., 204:125-139; Carter, P., et al., (1986) Nucl. Acids Res. 10:6487), cassette mutagenesis (Wells, J. A., et al., (1985) Gene 34:315), and restriction selection mutagenesis (Wells, J. A., et al., (1986) Philos. Trans. R. Soc., London Ser. A, 317:415).

In a preferred embodiment of the invention, the sequence of a GASP coding region is used as a guide to design a synthetic nucleic acid molecule encoding the GASP polypeptide that can be incorporated into a vector of the present invention. Methods for constructing synthetic genes are well-known to those of skill in the art. See, e.g., Dennis, M. S., Carter, P. and Lazarus, R. A. (1993) Proteins: Struct. Funct. Genet., 15:312-321. Expression and purification methods are described below in connection with the nucleic acids, vectors and host cells of the invention.

C. Uses of GASP Polypeptides

The GASP polypeptides of the invention are useful in a variety of research and therapeutic applications. The discovery of GASP polypeptides that modulate agonist-induced downregulation will facilitate studies aimed at elucidating the series of molecular events underlying these phenomena. In other research applications, the GASP polypeptides of the invention can be used in screening assays (see below) to identify additional molecules that inhibit or enhance agonist-induced downregulation. In addition, GASP polypeptides can be used as standards in immunoassays to detect the presence of GASP polypeptides in a biological sample.

GASP polypeptides can also be used therapeutically to treat a conditions that can be ameliorated by modulating agonist-induced downregulation. For example, as noted above with respect to the methods for modulating downregulation, the inhibition of agonist-induced downregulation can improve and/or prolong responsiveness to drugs or other agents whose effects are mediated by the receptor. Alternatively, the enhancement of agonist-induced downregulation can reduce unwanted biological responses to agents whose effects are mediated by the receptor. Pharmaceutical compositions containing the polypeptides of the invention are described in greater detail below.

Delta Opioid Receptor Polypeptides

A. Types and Production of DOR Polypeptides

The polypeptides of the invention also include delta opioid receptor (DOR) polypeptides A DOR polypeptide of the invention has the following features:

(1) the DOR polypeptide comprises a DOR amino acid sequence, i.e., an amino acid sequence that has at least about 70% identity to DOR SEQ ID NO:3 over a comparison window of at least 15 contiguous amino acids;

(2) the DOR amino acid sequence defines a peptide that specifically binds GASP SEQ ID NO:2 (GASP1) or GASP SEQ ID NO:6 (GASP2); and

(3) the DOR polypeptide does not, however, comprise more than about 50 contiguous amino acids of the delta opioid receptor. DOR SEQ ID NO:3 has the following amino acid sequence (given in single-letter amino acid code):

### GASP Binding Domain of DOR

SLNPVLYAFLDENFKRCFRQLCRTPCGRQEPGSLRRPRQATTRERVTACTPSDGPGG GAA

SEQ ID NO:3.

Percent identity can, for example, be determined by a sequence alignment performed using BLASTP with default parameters set to measure 70% identity. In variations of this embodiment, the percent identity is 80, 90, 95, 96, 97, 98, 99, or 100 percent. The invention also encompasses polypeptides wherein the percent identities noted above are found over a comparison window of at least 20, 30, or 40 or more contiguous amino acids. In various embodiments, the amino acid sequence includes an amino acid subsequence of at least about 15, 20, 24, 30, 35, 40, 45 or more amino acids, of DOR SEQ ID NO:3. In one embodiment, that amino acid sequence is DOR SEQ ID NO:3.

Generally, the DOR amino acid sequence defines a peptide that specifically binds to GASP SEQ ID NO:2 (GASP1) or GASP SEQ ID NO:6 (GASP2) with a higher affinity than MOR and, preferably, an affinity in approximately the same range (e.g., no more than about 5-fold less, preferably no more than about 2-fold less) as wild-type DOR.

The DOR amino acid sequence can be derived from any DOR polypeptide from any organism. DOR amino acid sequences useful in the invention are generally those derived from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. In particularly preferred embodiments, the DOR amino acid sequence is derived from a human polypeptide.

The DOR amino acid sequence can be a wild-type amino acid sequence or an amino acid sequence variant of the corresponding region of a wild-type polypeptide. Preferred DOR polypeptides generally include a wild-type DOR amino acid sequence or a DOR amino acid sequence containing conservative amino acid substitutions, as defined above.

In addition to the amino acid sequences described above, DOR polypeptides of the invention can include other amino acid sequences, including those from heterologous proteins. Accordingly, the invention encompasses fusion polypeptides in which the above-discussed amino acid sequence is fused, at either or both ends, to amino acid sequence(s) from one or more heterologous proteins. Examples of additional amino acid sequences often incorporated into proteins of interest include a signal sequence, which facilitates purification of the protein, and an epitope tag, which can be used for immunological detection or affinity purification.

DOR polypeptides of the invention can be otherwise modified to produce derivatives that retain the above-described functions, namely specific binding to GASP SEQ ID NO:2 (GASP1) or GASP SEQ ID NO:6 (GASP2). In preferred embodiments, the modified polypeptides have an activity that is about 0.1 to about 0.01-fold that of the unmodified forms. In more preferred embodiments, the modified polypeptides have an activity that is about 0.1 to about 1-fold that of the unmodified polypeptides. In even more preferred embodiments, the modified polypeptides have an activity that is greater than the unmodified polypeptides.

DOR polypeptides of the invention can be produced by any available technique, such as the synthetic and recombinant techniques discussed above with respect to GASP polypeptides.

B. Uses of DOR Polypeptides

The DOR polypeptides of the invention are useful in a variety of applications. As described in Example 1, DOR polypeptides can be used to identify GASP polypeptides, e.g., from additional organisms. In addition, DOR polypeptides can be employed to bind GASP polypeptides, in vitro or in vivo, for detection or to inhibit the binding interaction between GASP and G protein-coupled receptors that specifically bind to GASP. Alternatively, DOR polypeptides can be used in screening assays (see below) to identify additional molecules that inhibit or enhance agonist-induced downregulation. In addition, DOR polypeptides can be used as standards in immunoassays to detect the presence of DOR in a biological sample.

DOR polypeptides can also be used therapeutically to treat a conditions that can be ameliorated by inhibiting agonist-induced downregulation, thereby improving and/or prolonging responsiveness to drugs or other agents whose effects are mediated by the delta opioid receptor. Pharmaceutical compositions containing the polypeptides of the invention are described in greater detail below.

Polynucleotides, Vectors, and Host Cells

The invention also provides a polynucleotide encoding a polypeptide of the invention, a vector including this polynucleotide, and a host cell including the vector.

A. Polynucleotides

Polynucleotides of the invention include a portion that encodes a GASP or DOR polypeptide. As noted above, the encoded GASP or DOR amino acid sequence can be a wild-type sequence or a variant sequence. Where the GASP or DOR amino acid sequence is a wild-type sequence, the nucleotide sequence encoding this sequence can be a wild-type nucleotide sequence or one containing “silent” mutations that do not alter the amino acid sequence due to the degeneracy of the genetic code. For example, if the polynucleotide is intended for use in expressing the encoding polypeptide, silent mutations can be introduced by standard mutagenesis techniques to optimize codons to those preferred by the host cell.

In some applications, it is advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH2—NH—O—CH2, CH2—N(CH3)—O—CH2 (known as the methylene(methylimino) or MMI backbone) and CH2—O—N(CH3)—CH2, CH2—N(CH3)—N(CH3)—CH2, and O—N(CH3)—CH2—CH backbones (where phosphodiester is O—P—O—CH2). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

Those of skill in the art understand that polynucleotides complementary to the coding strand of polynucleotides of the invention can be employed to inhibit expression of the polypeptides of the invention, which may be of interest for research or therapeutic purposes. Accordingly, the nucleic acids of the invention include such “antisense polynucleotides,” and the phrase “polynucleotide encoding a polypeptide of the invention” is intended to include such antisense molecules.

B. Vectors

A polynucleotide of the present invention can be incorporated into a vector for propagation and/or expression in a host cell. Such vectors typically contain a replication sequence capable of effecting replication of the vector in a suitable host cell (i.e., an origin of replication) as well as sequences encoding a selectable marker, such as an antibiotic resistance gene. Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art.

If the vector is intended for expression of a polypeptide, the vector includes one or more control sequences capable of effecting and/or enhancing the expression of an operably linked polypeptide coding sequence. Control sequences that are suitable for expression in prokaryotes, for example, include a promoter sequence, an operator sequence, and a ribosome binding site. Control sequences for expression in eukaryotic cells include a promoter, an enhancer, and a transcription termination sequence (i.e., a polyadenylation signal).

An expression vector according to the invention can also include other sequences, such as, for example, nucleic acid sequences encoding a signal sequence or an amplifiable gene. A signal sequence can direct the secretion of a polypeptide fused thereto from a cell expressing the protein. In the expression vector, nucleic acid encoding a signal sequence is linked to a polypeptide coding sequence so as to preserve the reading frame of the polypeptide coding sequence. The inclusion in a vector of a gene complementing an auxotrophic deficiency in the chosen host cell allows for the selection of host cells transformed with the vector.

A vector of the present invention is produced by linking desired elements by ligation at convenient restriction sites. If such sites do not exist, suitable sites can be introduced by standard mutagenesis (e.g., site-directed or cassette mutagenesis) or synthetic oligonucleotide adaptors or linkers can be used in accordance with conventional practice.

Viral vectors are of particular interest for use in delivering polynucleotides of the invention to a cell or organism, followed by expression of the encoded protein, i.e., “gene therapy” when performed to ameliorate a pathological condition. For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous genes into most mammalian cells.

Adeno-associated virus (AAV)-based vectors used to transduce cells with target nucleic acids, e.g., in the in vitro production of nucleic acids and peptides, and in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

C. Host Cells

The present invention also provides a host cell containing a vector of this invention. A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, plant cells, and phage, as well as higher eukaryotic cells (such as human embryonic kidney cells and other mammalian cells). Host cells according to the invention include cells in culture and cells present in live organisms, such as transgenic plants or animals or cells into which a gene therapy vector has been introduced.

A vector of the present invention is introduced into a host cell by any convenient method, which will vary depending on the vector-host system employed. Generally, a vector is introduced into a host cell by transformation (also known as “transfection”) or infection with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

Recombinant Production Methods

Host cells transformed with expression vectors can be used to express the polypeptides encoded by the polynucleotides of the invention. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

Compositions

A. In General

For research and therapeutic applications, the GASP and DOR polypeptides of the invention or polynucleotides encoding them are preferably formulated for administration to cells, tissues, or organisms. These compositions are generally formulated to deliver GASP or DOR polypeptides to a target site in an amount sufficient to modulate (inhibit or enhance) agonist-induced downregulation of a G protein-coupled receptor. In one embodiment, the compositions also include an agonist of the G protein-coupled receptor, generally in an amount sufficient to activate G protein-coupled receptors at the target site.

B. Compositions Containing GASP and DOR Polypeptides

The invention provides compositions, including pharmaceutical compositions, containing a polypeptide of the invention. The compositions optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™. Pluronics™, and PEG). In one embodiment, the pharmaceutically acceptable carrier is an aqueous pH-buffered solution.

Preferred embodiments include sustained-release pharmaceutical compositions. An exemplary sustained-release composition has a semipermeable matrix of a solid hydrophobic polymer to which the polypeptide is attached or in which the polypeptide is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Exemplary sustained release compositions include polypeptides attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped polypeptide. Liposomes are small vesicles composed of various types of lipids, phospholipids, and/or surfactants. These components are typically arranged in a bilayer formation, similar to the lipid arrangement of biological membranes. Liposomes containing polypeptides are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions can also include a polypeptide adsorbed onto a membrane, such as a silastic membrane, which can be implanted, as described in International Publication No. WO 91/04014.

Pharmaceutical compositions of the invention can be stored in any standard form, including, e.g., an aqueous solution or a lyophilized cake. Such compositions are typically sterile when administered to recipients. Sterilization of an aqueous solution is readily accomplished by filtration through a sterile filtration membrane. If the composition is stored in lyophilized form, the composition can be filtered before or after lyophilization and reconstitution.

C. Compositions Containing Polynucleotides Encoding GASP and DOR Polypeptides

The invention provides compositions, including pharmaceutical compositions, containing a polynucleotide encoding the polypeptide of the invention. Such compositions optionally include other components, as for example, a storage solution, such as a suitable buffer, e.g., a physiological buffer. In a preferred embodiment, the composition is a pharmaceutical composition and the other component is a pharmaceutically acceptable carrier as described above.

In preferred embodiments, compositions containing polynucleotides of the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with the polynucleotides of the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above for polypeptides.

In another embodiment, polynucleotides are complexed to dendrimers, which can be used to transfect cells. Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimes and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides of the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides. In a preferred embodiment, therapeutic compositions of the invention include polynucleotides combined with lipids, as described above.

Compositions containing polynucleotides can be stored in any standard form, including, e.g., an aqueous solution or a lyophilized cake. Such compositions are typically sterile when administered to cells or recipients. Sterilization of an aqueous solution is readily accomplished by filtration through a sterile filtration membrane. If the composition is stored in lyophilized form, the composition can be filtered before or after lyophilization and reconstitution.

Anti-GASP Antibodies

The invention includes an antibody and an antiserum that specifically recognizes a GASP polypeptide of the invention. The invention encompasses polyclonal and monoclonal anti-GASP antibodies. Polyclonal antibodies are raised by injecting (e.g. subcutaneous or intramuscular injection) antigenic polypeptides into a suitable non-human mammal (e.g. a mouse or a rabbit). Generally, the polypeptide used to raise anti-GASP should induce production of high titers of antibody with relatively high affinity for GASP, and, in particular, for the GASP domain that specifically binds to G protein-coupled receptors.

If desired, the immunizing polypeptide may be conjugated to a carrier protein by conjugation using techniques that are well known in the art. Commonly used carriers include keyhole limpet hemocyanin (KLH), thyroglobulin, bovine serum albumin (BSA), and tetanus toxoid. The conjugate is then used to immunize the animal.

The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal, as well as monoclonal, antibodies see, for example, Coligan, et al. (1991) Unit 9, Current Protocols in Immunology, Wiley Interscience).

For many application, monoclonal anti-GASP antibodies are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

It is also possible to evaluate a mAb to determine whether it has the same specificity as a mAb described herein without undue experimentation by determining whether the mAb being tested prevents the described mAb from binding a target polypeptide. If the mAb being tested competes with the mAb described herein, it is likely that the two monoclonal antibodies bind to the same or a closely related epitope. Still another way to determine whether a mAb has the specificity of a mAb described herein is to preincubate the mAb described herein with an antigen with which it is normally reactive, and determine if the mAb being tested is inhibited in its ability to bind the antigen. Such inhibition indicates that the mAb being tested has the same, or a closely related, epitopic specificity as the mAb described herein.

As used herein, the term “antibody” encompasses antigen-binding antibody fragments, e.g., single chain antibodies (scFv or others), which can be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pill fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20-fold-1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural VH and VL repertoires present in human peripheral blood lymphocytes are isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al.

Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be prepared by any of a number of commercial services (e.g., Berkeley antibody laboratories, Bethyl Laboratories, Anawa, Eurogenetec, etc.).

Methods of Screening for Agents that Modulate Agonist-Induced Down-Regulation of G Protein-Coupled Receptors

The role of GASP polypeptides in agonist-induced downregulation of G protein-coupled receptors makes the GASP-receptor interaction an attractive target for agents that modulate agonist-induced downregulation of G protein-coupled receptors. Accordingly, the invention provides prescreening and screening methods aimed at identifying agents that either inhibit or enhance receptor downregulation. The prescreening/screening methods of the invention are generally, although not necessarily, carried out in vitro. Accordingly, screening assays are generally carried out, for example, using purified or partially purified components (GASP polypeptides or polynucleotides, G protein-coupled receptors, etc.), in cell lysates, in cultured cells, or in a biological sample.

A. Prescreening Based on Binding to GASP Polypeptides or Polynucleotides

The prescreening methods are based on screening test agents for specific binding, either to a GASP polypeptide or polynucleotide or to a G protein-coupled receptor. Agents that specifically bind to GASP polypeptides have the potential to disrupt the GASP-receptor interaction that promotes downregulation of G protein-coupled receptors that are downregulated by this mechanism. Accordingly, agents that specifically bind to GASP polypeptides, and in particular those that compete with the G protein-coupled receptor of interest for binding to GASP polypeptides, are candidate inhibitors of agonist-induced downregulation.

In one embodiment, therefore, a prescreening method of the invention entails contacting a test agent with a GASP polypeptide that specifically binds the G protein-coupled receptor of interest and modulates agonist-induced down-regulation of the receptor. Specific binding of the test agent to the GASP polypeptide is then detected. Suitable GASP polypeptides include an amino acid sequence that has at least about 70% identity to GASP SEQ ID NO:2 (GASP1) or GASP SEQ ID NO:6 (GASP2) over a comparison window of at least 15 contiguous amino acids. In a variation of this embodiment, the test agent is contacted with the GASP polypeptide in the presence of the G protein-coupled receptor or a fragment thereof that is capable of specifically binding to the GASP polypeptide. Specific test agent-GASP binding provides a measure of the ability of the test agent to compete with the G protein-coupled receptor of interest for binding to GASP polypeptides.

Agents that specifically bind to GASP polynucleotides have the potential to decrease or increase the expression of GASP polypeptides, which can inhibit or enhance, respectively, downregulation of G protein-coupled receptors. Therefore, agents that specifically bind to GASP polynucleotides are candidate modulators of GASP expression and, ultimately, GASP-mediated receptor downregulation. Thus, in an alternative embodiment, the test agent can be contacted with a polynucleotide encoding the GASP polypeptide to screen for agents that affect GASP expression, followed by detection of specific binding of the test agent to the GASP polynucleotide.

Such prescreening is generally most conveniently accomplished with a simple in vitro binding assay. Means of assaying for specific binding of a test agent to a polypeptide or polynucleotide are well known to those of skill in the art. In preferred binding assays, the GASP polypeptide of polynucleotide is immobilized and exposed to a test agent (which can be labeled), or alternatively, the test agent(s) are immobilized and exposed to the GASP polypeptide or polynucleotide (which can be labeled). The immobilized moiety is then washed to remove any unbound material and the bound test agent or bound GASP polypeptide or polynucleotide is then detected. To prescreen large numbers of test agents, high throughput assays are generally preferred. Various prescreening formats are discussed in greater detail below.

B. Prescreening Based on Binding to G Protein-Coupled Receptor Polypeptides

Prescreening for agents that modulate agonist-induced downregulation of G protein-coupled receptors can also be carried out based on screening test agents for specific binding to a G protein-coupled receptor. In particular, agents that specifically bind to the G protein-coupled receptor have the potential to disrupt the GASP-receptor interaction that promotes downregulation of G protein-coupled receptors that are downregulated by this mechanism. Alternatively, agents that specifically bind to the G protein-coupled receptor could mimic the downregulation promoting effect of GASP polypeptides.

Therefore, the invention provides method of prescreening for an agent that inhibits or enhances agonist-induced down-regulation of a G protein-coupled receptor, wherein the method entails contacting a test agent with a G protein-coupled receptor, or fragment thereof. Specific binding of the test agent to the receptor or receptor fragment is then detected. Generally, the G protein-coupled receptor or receptor fragment useful in this method is capable of specific binding to a polypeptide having the amino acid sequence of GASP SEQ ID NO:2 (GASP1) or GASP SEQ ID NO:6 (GASP2). Exemplary receptors include the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

Prescreening based on binding to G protein-coupled receptor polypeptides is generally most conveniently carried out using a simple in vitro binding assay, as discussed above, with high throughput assays are being preferred for prescreening large numbers of test agents.

C. Screening Based on Levels of GASP Polypeptide or RNA

Test agents, including, for example, those identified in a prescreening assay of the invention can also be screened to determine whether the test agent affects the levels of GASP polypeptide or RNA. Agents that reduce these levels can potentially inhibit GASP-mediated downregulation of G protein-coupled receptors. Conversely, agents that increase these levels can potentially enhance GASP-mediated downregulation of G protein-coupled receptors.

Accordingly, the invention provides a method of screening for an agent that inhibits or enhances agonist-induced down-regulation of a G protein-coupled receptor in which a test agent is contacted with a cell. The cell contains a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2). The cell additionally contains a GASP polypeptide (e.g., one comprising SEQ ID NO:2 [GASP1] or SEQ ID NO:6 [GASP2]), or a species or allelic variant thereof. After contact with the test agent, the level of GASP polypeptide or RNA is determined to identify any test agents that affect these levels.

Cells useful in this screening method include those described above with respect to methods of modulating agonist-induced downregulation of G protein-coupled receptors. Cells that naturally express G protein-coupled receptors that are subject to GASP-mediated downregulation are typically, although not necessarily, employed in this screening method. Of particular interest are cells expressing one or more of the following receptors: the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

1. Sample Collection and Processing

As noted above, screening assays are generally carried out in vitro, for example, in cell lysates, in cultured cells, or in a biological sample. (e.g., whole blood, plasma, serum, synovial fluid, cerebrospinal fluid, bronchial lavage, ascites fluid, bone marrow aspirate, pleural effusion, urine, or tumor tissue) derived from an animal, preferably a mammal, and more preferably from a human. For ease of description, cell cultures and biological samples are referred to as “samples” below.

The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

2. Polypeptide-Based Assays

GASP polypeptide(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one embodiment, the GASP polypeptide(s) are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of GASP polypeptide(s) in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with antibodies that specifically bind the target polypeptide(s). Antibodies that specifically bind to the target polypeptide(s) and may be directly labeled or alternatively may be detected subsequently using labeled antibodies (e.g., labeled sheep anti-mouse antibodies) that specifically bind to a domain of the primary antibody.

In a preferred embodiment, the GASP polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case a GASP polypeptide). In preferred embodiments, the capture agent is an antibody.

Immunoassays also typically utilize a labeling agent to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the label agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled GASP polypeptide is added to the sample, and the sample is then contacted with a capture agent. The amount of labeled GASP polypeptide bound to the antibody is inversely proportional to the concentration of GASP polypeptide present in the sample.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies useful in these immunoassays include polyclonal and monoclonal antibodies, which can be produced, for example, as described above.

3. Polynucleotide Based Assays

Changes in GASP expression level can be detected by measuring changes in mRNA and/or a polynucleotide derived from the mRNA (e.g., reverse-transcribed cDNA, etc.).

a. Polynucleotide Sample

The polynucleotide sample is, in certain embodiments, isolated from a biological sample according to any of a number of methods well known to those of skill in the art. For example, methods of isolation and purification of polynucleotides are described in detail in by Tijssen ed., (1993) Chapter 3 of Laboratory Techniques in Biochemistry and Molecular Biology: Hybridization With Polynucleotide Probes, Part I. Theory and Polynucleotide Preparation, Elsevier, N.Y. and Tijssen ed.

In a preferred embodiment, “total” polynucleotide is isolated from a given sample using, for example, an acid guanidinium-phenol-chloroform extraction method, and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)).

Frequently, it is desirable to amplify the polynucleotide sample prior to assaying for expression level. One of skill in the art will appreciate that whatever amplification method is used, if a quantitative result is desired, care must be taken to use a method that maintains or controls for the relative frequencies of the amplified polynucleotides.

Methods of “quantitative” amplification are well known to those of skill in the art. For example, quantitative PCR involves simultaneously co-amplifying a known quantity of a control sequence using the same amplification primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

b. Hybridization-Based Assays

(i) Detection of Gene Transcripts

Methods of detecting and/or quantifying the transcript(s) of one or more GASP gene(s) (e.g. mRNA or cDNA made therefrom) using polynucleotide hybridization techniques are known to those of skill in the art (see Sambrook et al. supra). For example, the presence, absence, or quantity of a reverse-transcribed cDNA can be measured by Southern blot. Alternatively, in a Northern blot, mRNA is directly quantitated. In both cases, labeled probes are used to identify and/or quantify the target mRNA.

The probes used herein for detection of the GASP polynucleotides can be full-length or less than the full-length of these polynucleotides. Shorter probes are empirically tested for specificity. Preferably polynucleotide probes are 20 bases or longer in length. (See Sambrook et al. for methods of selecting polynucleotide probe sequences for use in polynucleotide hybridization.) Visualization of the hybridized probes allows the qualitative determination of the presence or absence of the GASP polynucleotide, and standard methods (such as, e.g., densitometry) can be used to quantify the level of the GASP polynucleotide.

(ii) Amplification-Based Assays

In still another embodiment, amplification-based assays can be used to measure GASP expression level. In such amplification-based assays, the target polynucleotide sequences act as template(s) in amplification reaction(s) (e.g., Polymerase Chain Reaction (PCR) or reverse-transcription PCR (RT-PCR)). In a quantitative amplification, the amount of amplification product is proportional to the amount of template in the original sample. Detailed protocols for quantitative PCR are provided in Innis et al. (1990) PCR Protocols, A Guide to Methods and Applications, Academic Press, Inc. N.Y.).

Other suitable amplification methods include, but are not limited to, ligase chain reaction (LCR) (see Wu and Wallace (1989) Genomics 4: 560; Landegren et al. (1988) Science 241: 1077; and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

c. Hybridization Formats and Conditions

(i) Array-Based Hybridization Formats

In one embodiment, the screening methods of this invention can be carried out in an array-based hybridization format. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly polynucleotide arrays can be produced according to a wide variety of methods well known to those of skill in the art. For example, in a simple embodiment, “low density” arrays can simply be produced by spotting (e.g. by hand using a pipette) different polynucleotides at different locations on a solid support (e.g. a glass surface, a membrane, etc.). This simple spotting, approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide microarrays. Synthesis of high density arrays is also described in U.S. Pat. Nos. 5,744,305; 5,800,992; and 5,445,934.

In one embodiment, the arrays used in this invention are arrays “probe” polynucleotides. These probes are then hybridized respectively with their “target” polynucleotides (e.g., mRNA derived from a biological sample). The arrays can be hybridized with a single population of sample polynucleotide or can be used with two differentially labeled collections (as with a test sample and a reference sample). Alternatively, the format can be reversed, such that polynucleotides from different samples (i.e., the target polynucleotides) are arrayed and this array is then probed with one or more probes, which can be differentially labeled.

Many methods for immobilizing polynucleotides on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, and the like. In addition, substances that form gels can be used. Such materials include, e.g., proteins (e.g., gelatins), lipopolysaccharides, silicates, agarose, and polyacrylamides.

In preparing the surface, any of a variety of different materials may be employed, particularly as laminates, to provide desirable properties. For example, proteins (e.g., bovine serum albumin) or mixtures of macromolecules (e.g., Denhardt's solution) can be employed to reduce non-specific binding, simplify covalent conjugation, or enhance signal detection. If covalent bonding between a compound and the surface is desired, the surface will usually be polyfunctional or be capable of being polyfunctionalized. Functional groups which may be present on the surface and used for linking polynucleotides can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, polynucleotides can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). In addition, polynucleotides are conveniently modified by introduction of various functional groups that facilitate immobilization (see, e.g., Bischoff (1987) Anal. Biochem., 164: 336-344; Kremsky (1987) Nucl. Acids Res. 15: 2891-2910).

Arrays can be made up of target elements of various sizes, ranging from 1 mm diameter down to 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

(ii) Other Hybridization Formats

A variety of other hybridization formats are known to those skilled in the art and suitable for use in the screening methods of the invention. Hybridization techniques are generally described in Hames and Higgins (1985) Polynucleotide Hybridization, A Practical Approach, IRL Press; Gall and Pardue (1969) Proc. Natl. Acad. Sci. USA 63: 378-383; and John et al. (1969) Nature 223: 582-587. Common hybridization formats include sandwich assays and competition or displacement assays.

The sensitivity of hybridization assays may be enhanced through use of a polynucleotide amplification system that multiplies the target polynucleotide being detected. Examples of such systems include the polymerase chain reaction (PCR) system and the ligase chain reaction (LCR) system. Other methods recently described in the art are the polynucleotide sequence based amplification (NASBAO, Cangene, Mississauga, Ontario) and Q Beta Replicase systems.

(iii) Hybridization Conditions

Polynucleotide hybridization simply involves providing a denatured probe and target polynucleotide under conditions where the probe and its complementary target can form stable hybrid duplexes through complementary base pairing. The polynucleotides that do not form hybrid duplexes are then washed away leaving the hybridized polynucleotides to be detected, typically through detection of an attached detectable label. Polynucleotides are generally denatured by increasing the temperature or decreasing the salt concentration of the buffer containing the polynucleotides, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In general, there is a tradeoff between hybridization specificity (stringency) and signal intensity. In a preferred embodiment, the wash is performed at the highest stringency that produces consistent results and that provides a signal intensity greater than approximately 10% of the background intensity. Hybridization can performed at low stringency to ensure hybridization and then subsequent washes are performed to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be included in the reaction mixture.

Methods of optimizing hybridization conditions are well known to those of skill in the art (see, e.g., Tijssen (1993) Laboratory Techniques in Biochemistry and Molecular Biology, Vol. 24: Hybridization With Polynucleotide Probes, Elsevier, N.Y.). In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

(iv) Labeling and Detection of Polynucleotides

In a preferred embodiment, the hybridized polynucleotides are detected by detecting one or more labels attached to the sample polynucleotides. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to the target (sample) polynucleotide(s) prior to, or after the hybridization. So-called “direct labels” are detectable labels that are directly attached to or incorporated into polynucleotide probes prior to hybridization. In contrast, so-called “indirect labels” typically bind to the hybrid duplex after hybridization. Often, the indirect label binds to a moiety that is attached to or incorporated into the polynucleotide probe prior to the hybridization. Thus, for example, the polynucleotide probe may be biotinylated before the hybridization. After hybridization, an avidin-conjugated fluorophore will bind the biotin bearing hybrid duplexes providing a label that is easily detected. For a detailed review of methods of labeling polynucleotides and detecting labeled hybridized polynucleotides see Laboratory Techniques in Biochemistry and Molecular Biology, Vol. 24: Hybridization With Polynucleotide Probes, P. Tijssen, ed. Elsevier, N.Y. (1993).

The labels may be incorporated by any of a number of means well known to those of skill in the art. Means of attaching labels to polynucleotides include, for example nick translation or end-labeling.

D. Screening Based on Level of Agonist-Induced Down-Regulation of G Protein-Coupled Receptors

The invention also provides a screening method based on determining the effect, if any, of a test agent on the level of agonist-induced downregulation of a G protein-coupled receptor. To screen for an effect on GASP-mediated downregulation, the method entails contacting the test agent with a cell containing a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2). The cell additionally contains a GASP polypeptide (e.g., one comprising SEQ ID NO:2 [GASP1] or SEQ ID NO:6 [GASP2]), or a species or allelic variant thereof. Agonist-induced downregulation is then measured by contacting the cell with sufficient agonist to activate the G protein-coupled receptor. Agonist-induced downregulation is then determined in the presence and absence (or presence of a lower amount) of test agent to determine whether the test agent modulated agonist-induced downregulation.

Agonist-induced downregulation can be determined by any of a variety of methods, including those described herein. For example, cell surface receptors can be measured and/or receptor proteolysis or localization in lysozomes can be determined. Alternatively, receptor downregulation can be determined indirectly by measuring desensitization of receptors after activation with an agonist. Desensitization can be determined, for example, by measuring a biological effect that is mediated by the receptor. (See FIG. 1 for examples of each of these methods.) Generally, it will be most convenient to measure cell surface receptors using a radio- or immunoassay. An exemplary radioligand binding assay useful for determining agonist-induced downregulation is described in Example 1. (See FIG. 1A and Reference 16.) Briefly, cells expressing the cell surface receptor of interest are incubated with a suitable agonist under conditions designed to provide a saturating concentration of agonist over the incubation period. After agonist treatment, the cells are recovered and assayed for radioligand binding. Cells that form monolayers can, for example, be collected with phosphate-buffered saline (PBS) supplemented with EDTA, followed by washing four times by centrifugation with 10 mL of warm (37° C.) PBS and one time by centrifugation with 10 mL of Krebs-Ringer HEPES buffer (KHRB: 110 mM NaCl, 5 mM KCl, 1 mM MgCl₂, 1.8 mM CaCl₂, 25 mM glucose, 55 mM sucrose, 10 mM HEPES, pH 7.3). Radioligand binding can then be carried out in 120 μL of KHRB containing equal amounts of washed cells (50-100 μg of protein). Incubations can be carried out, for example, for 30 minutes at room temperature. Cells can then be harvested and washed using vacuum filtration on glass fiber filters, followed by a determination of the radioligand bound to the filters.

Alternatively, radioligand binding can be assayed in membrane fractions. Reference 16, for example, describes such an exemplary assay of this type. In particular, after agonist treatment, cell monolayers are lifted with PBS containing 2 mM EDTA, washed twice by centrifugation (200×g for 5 minutes) with PBS, and lysed in 10 mM Tris-Cl, 2 mM EDTA, pH 7.4, containing a protease inhibitor mixture (leupeptin, aprotinin, pepstatin, an phenylmethylsulfonyl fluoride) followed by four passes using a tight-fitting Dounce homogenizer. Large particulates and nuclear material can then removed by centrifugation at 500×g for 5 minutes, and a membrane and cytosol fraction is collected. Binding assays are conveniently conducted in 120 μL of 25 mM Tris-Cl, 1 mM EDTA, pH 7.4. Assay tubes containing 50-100 μg of the membrane preparation (determined by the Bradford method, e.g., using reagents from Bio-Rad) and a suitable radioligand are incubated for 30 minutes at room temperature. Incubations are terminated by vacuum filtration through glass fiber filters (Packard Instruments) and repeated washes with ice-cold Tris-buffered saline, pH 7.4. Bound radioactivity is determined by scintillation counting (Scintiverse, Fisher) using a Beckman LS 6500 instrument.

E. Test Agent Databases

In a preferred embodiment, generally involving the screening of a large number of test agents, the screening method includes the recordation of any test agent that induces a difference in the level of GASP polypeptide or RNA in a database of agents that modulate agonist-induced downregulation.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

Test Agents Identified by Screening

When a test agent is found to reduce the level of GASP polypeptide or RNA or to reduce agonist-induced downregulation of a G protein-coupled receptor, a preferred screening method of the invention further includes selecting the test agent as an inhibitor of agonist-induced downregulation of the receptor. Conversely, when a test agent is found to increase the level of GASP polypeptide or RNA or to increase agonist-induced downregulation of a G protein-coupled receptor, a preferred screening method of the invention further includes selecting the test agent as an enhancer of agonist-induced downregulation of the receptor.

In both embodiments, methods of the invention optionally include combining the inhibitor or enhancer with a carrier, preferably pharmaceutically acceptable carrier, such as are described above. Generally, the concentration of inhibitor or enhancer is sufficient to inhibit or enhance, respectively, agonist-induced downregulation when the composition is contacted with a cell, as described above for the GASP or DOR-containing compositions and the methods of the invention for modulating agonist-induced downregulation. This concentration will vary, depending on the particular inhibitor/enhancer and specific application for which the composition is intended. As one skilled in the art appreciates the considerations affecting the formulation of a test inhibitor/enhancer with a carrier are generally the same as described above.

Methods of the invention can also include combining resultant test agent compositions with an agonist of the G protein-coupled receptor. Typically, the agonist concentration will be sufficient to activate the G protein-coupled receptor when the composition is contacted with a cell containing a suitable receptor. This activation would normally result in downregulation of the receptor, which is reduced or prevented in the presence of the test inhibitor. In compositions containing test agent enhancers in combination with an agonist, the agonist-induced downregulation is increased as compared to that observed upon agonist activation of the receptor in the absence of the test enhancer.

### EXAMPLE

The following example is offered to illustrate, but not to limit, the claimed invention.

### Example 1

### Post-Endocytic Sorting of G Protein-Coupled Receptors is Modulated by Interaction with the Novel Protein GASP1

### SUMMARY

Recycling of the mu opioid receptor (MOR) back to the plasma membrane following its endocytosis promotes rapid resensitization of signal transduction, whereas targeting of the structurally homologous delta opioid receptor (DOR) to lysosomes causes proteolytic downregulation. The carboxyl-terminal cytoplasmic domain contains a signal that distinguishes the endocytic sorting of opioid receptors. The following study identified a protein that binds preferentially to the cytoplasmic tail of DOR as a G protein coupled receptor associated sorting protein (GASP). Disruption of the DOR-GASP interaction through receptor mutation or overexpression of a dominant negative fragment of GASP inhibited receptor trafficking to lysosomes and promoted recycling to the plasma membrane. GASP also bound to the cytoplasmic tails of certain non-opioid G protein-coupled receptors (GPCRs), including the beta-2 adrenergic receptor (B2AR), but not to the V2 vasopressin receptor, which remains in endocytic vesicles for prolonged periods of time without trafficking to lysosomes. Disruption of a distinct protein interaction mediating rapid recycling of the B2AR caused endocytosed receptors to traffic to lysosomes in a GASP-dependent manner. Thus, GASP-family proteins modulate lysosomal sorting and functional downregulation of a variety of GPCRs.

Expression and Down-Regulation of DOR and MOR

To investigate the down-regulation of DOR and MOR, cells stably expressing MOR, DOR or D MOR (˜1-2 pmol/mg) were treated with agonist (10 μM DADLE) for 3 hours or left untreated. (See 18 and 45 for the DOR and MOR expression constructs, respectively.) Cells were then chilled on ice, washed extensively and total opioid radioligand binding sites were determined for each cell line (16). Epitope-tagged DOR (DOR-1 (13)) expressed in stably transfected HEK293 cells (14) exhibited pronounced downregulation within 3 hours of exposure to agonist (DADLE), whereas epitope-tagged MOR (MOR-1 (15)) expressed at similar levels did not (FIG. 1A) (16).

A biochemical assay that specifically measures the fate of surface-biotinylated receptors following their endocytosis (17) (12, 18) indicated that DOR but not MOR was rapidly proteolyzed after endocytosis (FIG. 1B, left panels). DORs treated for 90 minutes with agonist were concentrated in discrete areas of the cell where they colocalized with LAMP1 and LAMP2, membrane markers of late endosomes and lysosomes (FIG. 1C, left panels) (19). In contrast MORs, which exhibited little downregulation of ligand binding sites (FIG. 1A) or proteolysis of receptor protein (FIG. 1B), were localized in vesicles distributed throughout the cytoplasm (FIG. 1C, left panels note open arrows) and failed to colocalize substantially with LAMP1 and LAMP2 (FIG. 1C, right panels closed arrows) under these conditions.

DOR Tail Modulates DOR Sorting and Downregulation

Replacing the cytoplasmic tail of MOR with the corresponding sequence from the cytoplasmic tail of DOR strongly enhanced downregulation of ligand binding sites and post-endocytic proteolysis of a chimeric mutant MOR (D MOR for degrading MOR, FIGS. 1A and 1B, respectively) (12). Conversely, replacing the 30 C-terminal residues of the DOR tail with the corresponding 38-residue sequence derived from MOR inhibited post-endocytic proteolysis of a chimeric mutant DOR(R DOR for recycling DOR, FIG. 1B) (20). Thus, the DOR tail contains a signal that is not conserved in MOR and controls sorting of internalized receptors to lysosomes. This sorting event is functionally significant because it determined whether agonist-induced endocytosis caused functional resensitization or downregulation of opioid receptor-mediated signal transduction (FIG. 1D) (9, 21).

G Protein-Coupled Receptor Associated Sorting Protein

Because lysosomal sorting of DOR does not require post-translational phosphorylation (18) or ubiquitination (22) of the cytoplasmic tail, it was possible to identify protein(s) mediating DOR sorting using conventional interaction cloning. A yeast two hybrid screen of an HEK293 cell-derived cDNA library using the DOR tail domain (46) as bait yielded multiple clones corresponding to various carboxyl-terminal portions of a large predicted protein, whose mRNA is expressed in many tissues and is enriched in brain (23). A full-length cDNA (KIAA0443) encoding a 1395-residue predicted protein was obtained from the Kazusa DNA Research Institute (23).

Affinity chromatography using glutathion S transferase (GST)-fusion proteins confirmed that the cloned interacting protein bound specifically to the DOR tail and not to GST lacking the tail sequence (FIG. 1A) (24). This protein bound detectably but much more weakly to the cytoplasmic tail of MOR (FIG. 1A) and R DOR (not shown), consistent with a potential function in distinguishing the endocytic sorting of DOR and MOR. Therefore, this protein has been named “GASP1” for “G protein-coupled receptor-associated sorting protein-1.” Psi-BLAST searches conducted against the GenBank database indicated that GASP1 is a novel protein with both a rat and a murine homologue, and is homologous to several other human proteins of unknown function. Sequence homology to a non-mammalian protein was observed between the carboxyl-terminal regions of GASP1 and Vac8p, a yeast protein involved in late endocytic trafficking (26).

A rabbit polyclonal antibody was raised against the carboxyl-terminal 15 residues of GASP1 (27). This antibody recognized a major immunoreactive protein in immunoblots of (untransfected) HEK293 cell lysates, which co-electrophoresed with recombinant HA-tagged GASP1 protein expressed in HEK293 cells (FIG. 2B) and also had indistinguishable electrophoretic mobility from recombinant GASP1 produced by in vitro translation (FIG. 1A) (28, 29).

The antibody was used to assess whether endogenous GASP1 and DOR interacted in vivo. Although this antibody did not detectably stain endogenous GASP1 in fixed cells visualized by fluorescence microscopy, recombinant GASP1 tagged with HA or GFP localized throughout the cytoplasm and was not visualized in the nucleus (data not shown). A fraction of the endogenous GASP1 present in HEK293 cell lysates co-immunoprecipitated specifically with full length DOR but not with MOR expressed at similar levels (FIG. 2C, see legend for quantitation) (30). Conversely, DOR co-immunoprecipitated with endogenously expressed GASP1, confirming the occurrence and specificity of the receptor-GASP1 interaction in intact cells (FIG. 2D).

C-Terminal Fragment of G Protein-Coupled Receptor Associated Sorting Protein Modulates DOR Sorting and Downregulation

GST affinity chromatography was used to identify a carboxyl-terminal fragment of the GASP1 protein (cGASP1, corresponding to the C-terminal 497 residues of GASP1) that bound specifically to the DOR tail, consistent with the finding that several 2 hybrid hits contained this portion of GASP1 (FIG. 3A). cGASP1 bound to the DOR tail with an apparent affinity comparable to that of full length GASP1 (FIG. 3B) (24), as indicated by the similar fraction of GASP1 and cGASP1 recovered on beads when applied at similar concentrations and assayed by parallel GST binding (FIG. 2A, 3B and legend). A GFP-tagged version of cGASP1 stably overexpressed in HEK293 cells co-immunoprecipitated with wild type DOR but not MOR (FIG. 3C) (30), demonstrating in vivo that the specificity of the cGASP1-DOR interaction parallels that of the DOR-GASP1 interaction. cGASP1 was able to compete for binding of full length GASP1 to the DOR tail in vitro (FIG. 3D) (31). Furthermore, GFP-cGASP1, when highly overexpressed (estimated by immunoblotting to be approximately 40-fold over endogenous GASP1, see FIG. 3E, right panel), substantially reduced the amount of endogenous GASP1 recovered in DOR immunoprecipitates (FIG. 3E, left panel), suggesting that cGASP1 can function in intact cells as a dominant inhibitor of the interaction between endogenous GASP1 and DOR.

To begin to examine the role of GASP1 in mediating sorting of DOR in intact cells, an HA-tagged cGASP1 construct was expressed by transient transfection in cells stably expressing FLAG-tagged DOR. Trafficking of antibody labeled receptors was examined by fluorescence microscopy and dual color labeling was used to identify cGASP1-transfected cells in the cell population (19). Cells overexpressing cGASP1 showed robust internalization of the DOR in response to agonist, which was similar to that observed in adjacent cells in the specimen not expressing cGASP1 (FIG. 4A left panels). However, in cells expressing cGASP1, an increased amount of internalized receptors returned to the plasma membrane after agonist washout (FIG. 4A, right panel, open arrows). In contrast, internalized DOR remained predominantly in endocytic vesicles of adjacent cells not overexpressing cGASP1 (FIG. 4A, closed arrows). These results suggest that cGASP1 can function as a dominant inhibitor of post-endocytic sorting of DOR, consistent with the ability of cGASP1 to compete with full length/endogenous GASP1 for binding to DOR both in vitro (FIG. 3D) and in vivo (FIG. 3E).

A dominant negative effect of cGASP1 on lysosomal sorting of wild type DOR was supported by examining the localization of DOR relative to lysosomal markers in cells stably expressing both DOR and cGASP1. In these cGASP1-overexpressing cells, internalized DOR exhibited reduced colocalization with LAMP following 90 minutes treatment with agonist (FIG. 4B), compared to DOR in cells expressing only endogenous GASP1 (FIG. 1C) (19). In addition, DOR-containing endocytic vesicles visualized in cGASP1-overexpressing cells were still localized in a dispersed vesicular pattern following 90 minutes of agonist treatment, more closely resembling the distribution of MOR rather than DOR (compare DOR in FIG. 4B to FIG. 1C, DOR and MOR). The effects of cGASP1 were further examined using the biotin protection-degradation assay (12, 17, 18). Internalized DOR was proteolyzed almost completely in cells not overexpressing cGASP1 within 3 hours in the presence of agonist (FIG. 4C upper panel, and FIG. 1B), consistent with previous studies demonstrating extensive lysosomal proteolysis of receptors at this time point (6). However, in cells overexpressing cGASP1, a significant signal representing internalized DOR that had not been proteolyzed was detected under the same conditions (FIG. 4C, compare right lanes in upper and lower panels). Thus cGASP1 overexpression appeared to inhibit or delay the proteolysis of the internalized pool of DOR, without blocking the initial endocytosis of DOR from the plasma membrane (FIG. 4C, lane 4). Agonist-induced downregulation of DOR (measured by radioligand binding assay) was also detectably inhibited in cells overexpressing cGASP1 (FIG. 4E), and the extent of inhibition correlated with the fold overexpression of cGASP1. However ligand-induced proteolysis of epidermal growth factor (EGF) receptors, a distinct class of receptor tyrosine kinases that are expressed endogenously in these cells and traffic to lysosomes after agonist-induced endocytosis, was not was not detectably inhibited by cGASP1 overexpression (FIG. 4D) (32). These results suggest that overexpression of cGASP1 specifically inhibited sorting of the DOR GPCR to lysosomes without affecting the trafficking of a structurally distinct receptor that traverses a similar endocytic pathway.

The most dramatic effects on DOR trafficking and downregulation were observed in cell clones overexpressing cGASP1 at high levels (approximately 40-100-fold over that of the endogenous GASP1 protein) (FIG. 4E), suggesting that the dominant negative cGASP1 function is antimorphic. However, cGASP1 might have exerted its dominant negative effect on DOR sorting not because it blocked DOR binding to endogenous GASP1, but because it blocked DOR binding to a different protein or had other cellular effects. If this were the case, overexpression of full length GASP1, which also binds to DOR in vitro and in vivo, would inhibit DOR sorting to lysosomes. It was difficult to generate cell lines that stably overexpress full-length GASP1 at high levels. Several rounds of selection and subcloning produced a cell line that stably overexpressed a GFP-tagged version of full length GASP1 approximately 4-fold over endogenous GASP1 protein levels (FIG. 4G). This cell line had a doubling time three times that of cell clones of DOR-HEK293 cells expressing endogenous levels of GASP1 or overexpressing cGASP1 (not shown), suggesting that overexpression of full length GASP1 was not well tolerated. Nevertheless, in these cells, downregulation of DOR was not inhibited but, instead, was significantly enhanced (FIG. 4F). Thus, these results suggest that cGASP1 affects DOR trafficking by specifically inhibiting binding of DOR to endogenous GASP1, indicating that GASP1 is indeed a sorting protein that promotes trafficking of internalized DOR to lysosomes.

Interaction of G Protein-Coupled Receptor Associated Sorting Protein with Other Receptors

The ability of GASP1 to modulate the endocytic sorting of other (non-opioid) GPCRs was examined next. A limited survey using GST affinity chromatography indicated that cGASP1 interacted with the cytoplasmic tails of several catecholamine receptors, including the B2AR (FIG. 5A) and alpha-2B adrenergic receptor (not shown), a distinct catecholamine receptor that has been shown previously to undergo agonist-induced proteolysis (33), as well as the dopamine D4 receptor (FIG. 5A). Similar results were obtained using full length GASP1 (not shown). In contrast, cGASP1 (and GASP1, not shown) bound very weakly to the cytoplasmic tail of the V2 vasopressin receptor and, as shown above, also bound relatively weakly to the cytoplasmic tail of MOR (FIG. 5A). The lack of GASP1 interaction with the V2 receptor tail is consistent with the remarkable ability of these receptors to remain in endocytic vesicles for a prolonged period of time after endocytosis without undergoing any detectable downregulation (34).

The ability of GASP1 to bind to the B2AR tail in vitro was initially surprising, since the B2AR recycles efficiently after agonist-induced endocytosis and is relatively resistant to proteolysis in HEK293 cells (35). It has been shown previously that efficient recycling of the B2AR in these cells requires the actin cytoskeleton and a distinct set of protein interactions with the distal tip of the cytoplasmic tail (7). Adding a single alanine residue to the carboxyl terminus of the B2AR specifically disrupts this “recycling protein” interaction, creating a mutant B2AR (B2AR-Ala) that is sorted to lysosomes after agonist-induced endocytosis (7) (and see this study FIG. 5C) much like DOR (FIG. 1B). Hence, it was possible that cGASP1 might also interact in vivo with the B2AR-Ala mutant receptor and modulate its sorting to lysosomes. Consistent with this, GFP-cGASP1 co-immunoprecipited with B2AR-Ala (FIG. 5B) (30). Furthermore, overexpression of GFP-cGASP1 inhibited degradation of internalized B2AR-Ala (FIG. 5C), providing evidence for a functional role of the GASP1 protein interaction in lysosomal sorting of a non-opioid GPCR.

### CONCLUSION

GASP is a novel cytoplasmic protein that interacts selectively with a subset of GPCRs and modulates the sorting of internalized receptors to lysosomes. Differences in the post-endocytic sorting of DOR and MOR correlate with differences in the apparent affinity of GASP binding to the cytoplasmic tail, and mutations of the receptor tail that affect GASP binding have a corresponding effect on opioid receptor trafficking. The effects of GASP are not limited to opioid receptors because a mutant B2AR missing a distinct recycling signal present in the cytoplasmic tail (7, 36) is sorted to lysosomes in a GASP-dependent manner.

Thus, the postendocytic sorting of certain GPCRs appears to be regulated by multiple mechanisms, which have opposing effects on the sorting of receptors between divergent downstream membrane pathways. Certain GPCRs are targeted to lysosomes by a mechanism involving ubiquitination of lysine residues present in the carboxyl-terminal cytoplasmic domain. Previous studies have implicated ubiquitination of DOR specifically in a proteosome-dependent mechanism of receptor degradation (37) but not in receptor proteolysis by lysosomes (38), and our in vitro studies indicate that GASP binds to non-ubiquitinated receptor tails. Thus, GASP is involved in a distinct, noncovalent mechanism that promotes sorting of certain GPCRs to lysosomes. The ability of GASP to also modulate endocytic trafficking of the B2AR, whose trafficking to lysosomes has been shown recently to require ubiquitination (39) and whose recycling to the plasma membrane is further controlled by a distinct protein interaction with the cytoplasmic tail (7, 40), suggests that there may exist multiple mechanisms controlling the postendocytic membrane trafficking of certain mammalian GPCRs. Such complexity may provide redundant mechanisms for assuring the appropriate attenuation of receptor-mediated signals yet, at the same time, allow for the considerable diversity and flexibility of GPCR regulation observed in complex multicellular organisms (reviewed in (41).

In addition to their role in regulating GPCR signaling in response to physiological stimuli, specific protein interactions controlling post-endocytic sorting of GPCRs have therapeutic implications. For example, a compound that disrupted the interaction of DOR with GASP is expected to block downregulation and promote functional resensitization of receptors after ligand-induced activation and endocytosis. As a result, the signaling capacity of DOR in response to prolonged or repeated exposure to endogenously produced opioid peptides or exogenously administered alkaloid agonist drugs could be enhanced. Previous studies have suggested that opiate agonists that specifically activate delta but not mu opioid receptors are poor analgesics compared to compounds that activate both mu and delta or mu alone (42). While differences in the distribution of individual opioid receptor subtypes in neural tissue (43) and/or the distribution of the delta receptors in the circuits responsible for analgesia could contribute to this phenomenon, it is possible that GASP-mediated regulation of opioid receptor membrane trafficking plays an important role in determining receptor-specific differences in drug response. Thus, manipulating GASP-mediated downregulation of DOR, for example, is one avenue for developing novel analgesic compounds or improving analgesic responses to existing opiate drugs.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. S. S. Ferguson, J. Zhang, L. S. Barak, M. G. Caron, Life Sciences 62, 1561-5 (1998).

2. O. B. Goodman, Jr. et al., Adv Pharmacol 42, 429-33 (1998).

3. J. Trejo, S. R. Coughlin, J Biol Chem 274, 2216-24. (1999).

4. R. J. Lefkowitz, J. Pitcher, K. Krueger, Y. Daaka, Adv Pharmacol 42, 416-20 (1998).

5. A. W. Gagnon, L. Kallal, J. L. Benovic, J Biol Chem 273, 6976-81. (1998).

6. P. 1. Tsao, M. von Zastrow, J Biol Chem 275, 11130-40 (2000).

7. T. T. Cao, H. W. Deacon, D. Reczek, A. Bretscher, M. von Zastrow, Nature 401, 286-90. (1999).

8. J. Zhang et al., Proceedings of the National Academy of Sciences of the United States of America 95, 7157-62 (1998).

9. J. L. Whistler, M. von Zastrow, Proc Natl Acad Sci USA 95, 9914-9 (1998).

10. P. Y. Law, H. H. Loh, J Pharmacol Exp Ther 289, 607-24. (1999).

11. T. Koch et al., J Biol Chem 273, 13652-7. (1998).

12. A. K. Finn, J. L. Whistler, Neuron 32, 829-39. (2001).

13. C. J. Evans, D. E. Keith, Jr., H. Morrison, K. Magendzo, R. H. Edwards, Science 258, 1952-5. (1992).

14. Human embryonic kidney (HEK) 293 cells (ATCC) were grown in Dulbecco's modified Eagle's medium supplemented with 10% fetal bovine serum. Cells were transfected using calcium phosphate co-precipitation. Stably transfected cells were isolated following selection on 0.5% geneticin (Life technologies) or 0.2% Zeocin (Invitrogen).

15. D. L. Kaufman et al., Journal of Biological Chemistry 270, 15877-15883 (1995).

16. Ligand binding was carried out as previously described (12).

17. Biotin protection-degradation assay for proteolysis of surface-biotinylated receptors following endocytosis: Cells were grown to 80% confluency washed 2 times with cold phosphate buffered saline (PBS) then incubated in 3 μg/ml cleavable biotin (Pierce) in PBS at 4° C. for 30 minutes with gentle agitation. Cells were washed 2 times with Tris buffered saline and placed back into medium for treatment. Cells labeled “100% biotinylated” were left on ice in PBS. Cells labeled “100% stripped” were also left on ice in PBS then stripped as described below. Cells were treated with 5 μM agonist for 30 minutes or 3 hours, washed 2 times with cold PBS and the remaining cell surface biotinylated receptors were stripped in 50 mM glutathione, 0.3 M NaCl, 75 mM NaOH, 1% fetal bovine serum at 4° C. for 30 minutes. Glutathione was quenched with a 20 minute wash of PBS with 50 mM iodoacetamide, 1% bovine serum albumin. Cells were extracted in 0.1% Triton X-100, 150 mM NaCl, 25 mM KCl, 10 mM Tris-HCl pH 7.4 containing 1 μM leupeptin, 1 μM pepstatin A, 1 μM aprotinin, 2.5 μM Pefabloc SC, and cell debris was removed by centrifugation at 10,000×g for 10 minutes at 4° C. Receptors were immunoprecipitated using M2 antibody (Sigma), rabbit anti mouse linker (Jackson Immunoresearch) and protein A sepharose (Pharmacia) overnight. Precipitates were extensively washed, and deglycosylated for 2 hours at 37° C. with PNGase (NEB). Proteins were denatured in SDS sample buffer with no reducing agent and separated by SDS-PAGE. Proteins were transferred to nitrocellulose and the membrane blocked in Tris buffered saline containing 0.1% Tween and 5% nonfat milk for 1 hour. Biotinylated proteins were visualized by incubating with the Vectastain ABC immunoperoxidase reagent (Vector laboratories), followed by development with ECL reagents (Amersham).

18. J. L. Whistler, P. Tsao, M. von Zastrow, J Biol Chem 276, 34331-8. (2001).

19. Assay of colocalization of opioid receptors with LAMP 1 and LAMP2: Cells were grown on coverslips and incubated in media containing 3.5 μg/ml M1 anti-FLAG antibody (Sigma) antibody (to visualize FLAG-tagged DOR). Cells were then treated as described with 5 μM agonist for 30 minutes, followed by 30 minutes with 10 μM antagonist, and fixed using 4% formaldehyde in phosphate buffered saline. For visualization of receptor and green fluorescent protein (GFP)-tagged GASP1, cells were then incubated with Texas Red conjugated donkey anti-mouse antibody (Jackson Immunoresearch). For visualization of receptor and hemagglutinin (HA)-tagged GASP1, cells were first incubated with HA-11 (Covance) and an IgG2b-specific rabbit anti-mouse linker antibody (Zymed) followed by incubation with IgG1 specific FITC conjugated goat-anti mouse (Boeringer Mannheim) and a Texas red conjugated donkey anti-rabbit (Jackson Immunoresearch) secondary antibodies. For co-localization of LAMP and receptor, cells were fed antibody as above and treated with agonist for 90 minutes, fixed, permeabilized and incubated with anti-LAMP1 and LAMP2 antibodies (IgG1) and a rabbit anti-mouse IgG2b antibody (Zymed). Cells were then washed and incubated with Texas Red-conjugated donkey anti-rabbit (Jackson Immunoresearch) and IgG1-specific FITC-conjugated donkey anti-mouse (Boeringer Mannheim) antibodies.

20. R DOR was constructed by replacing the 30 C-terminal residues of the DOR with the 38 corresponding residues of the MOR. The C-terminal 170 amino acids of R DOR (in single-letter amino acid code) is as follows:

IDENFKRCFRQLCRTPCGRQEPQNSARIRQNTREHPSTANTVDRTNHQLENLEAETA PLP

SEQ ID NO:4

21. Membrane adenylyl cyclase assays were performed as previously described (9).

22. M. Tanowitz, M. von Zastrow (in preparation).

23. M. Suyama, T. Nagase, O. Ohara, Nucleic Acids Res 27, 338-9. (1999).

24. Affinity chromatography was carried out as previously described (25).

25. U. Klein, M. T. Ramirez, B. K. Kobilka, M. von Zastrow, J Biol Chem 272, 19099-102. (1997).

26. Y. X. Wang, N. L. Catlett, L. S. Weisman, J Cell Biol 140, 1063-74. (1998).

27. The GASP1 antibody was raised commercially by Zymed to the 15 C-terminal residues of GASP1 using standard methods.

28. The GASP1 sequence encodes an acidic protein with a predicted mass of 156.8 kD. In vitro transcription/translation of this clone yielded a protein with an apparent mass of 190 kD when resolved by SDS PAGE in Tris-Glycine buffer and 170 kD in MOPS buffer.

29. M. G. Peterson, N. Tanese, B. F. Pugh, R. Tjian, Science 248, 1625-30. (1990).

30. Assay for coimmunoprecipitation of cGASP1 with opioid receptors: Cells were grown to confluency, washed 2 times with PBS and lysed in 0.1% Triton X-100, 150 mM NaCl, 25 mM KCl, 10 mM Tris-HCl pH 7.4 containing 1 μM leupeptin, 1 μM pepstatin A, 1 μM aprotinin, 2.5 μM Pefabloc SC, and cell debris was removed by centrifugation at 10,000×g for 10 minutes at 4° C. An aliquot of lysate was removed for GASP1 control blot. Lysate was incubated with M2 affinity resin (Sigma) overnight. Pellets were extensively washed and incubated with PNGase (NEB) for 2 hours at 37° C. to degycosylate receptor protein. Samples were eluted in SDS sample buffer. For co-immunoprecipitation with endogenous GASP1, one fourth of eluate was separated by SDS-PAGE on a 12% gel (receptor blot), three quarters on a 7% gel (GASP1 blot). For co-immunoprecipitation with cGASP1 eluate was run on a 12% gel that was later cut and blotted separately for cGASP1 and receptor. Proteins were transferred to nitrocellulose. GASP1 blots were incubated for 2 hours with rabbit anti-GASP1 (1:4000). cGASP1 blots were incubated with rabbit anti-GFP antibodies for 2 hours (1:200) (Clonetech). Both were followed by 1 hour with horseradish peroxidase (HRP)-conjugated anti-rabbit antibody (NEB) (1:3000), and visualized with ECL plus (Amersham). Receptor blots were incubated with biotinylated M2 (1:250) (Sigma) for two hours, followed by visualization with Vectastain ABC reagents (Vector) and ECL plus. HA-11 (Covance) was used for blotting at 1:1000 for 2 hours for HA-GASP1 blots.

31. DOR-GASP1 binding assay: MBP-DOR cytoplasmic tail and MBP-lacZ fusion proteins were expressed and purified on amylose resin. Full length GASP1 probe was generated by in vitro transcription/translation as previously described (25) and incubated with 15 μg MPB-fusion protein. For competition, GST-cGASP1 was bacterially expressed, purified on glutathione agarose and the purified protein was eluted with glutathione and quantified. Eluted GST-cGASP1 was added to the MBP-DOR slurry just before addition of in vitro translated full length GASP1 probe.

32. EGFR assay: Cells expressing only endogenous GASP1 and cells overexpressing GFP-cGASP1 (>40×) were grown to 80% confluency and treated with 5 μM EGF in DMEM for the indicated times or left untreated. Cells were washed with PBS, EGF receptor was immunoprecipitated with Rabbit anti EGFR-affinity resin (Santa Cruz Biotechnology), separated by SDS page and immunoblotted with goat anti-EGFR antibodies (Santa Cruz Biotechnology), followed by HRP-conjugated anti-goat secondary (Jackson Immunoresearch) and development with ECL reagents (Amersham).

33. D. A. Heck, D. B. Bylund, J Pharmacol Exp Ther 282, 1219-27. (1997).

34. G. Innamorati, H. M. Sadeghi, N. T. Tran, M. Bimbaumer, Proc Natl Acad Sci USA 95, 2222-6. (1998).

35. M. von Zastrow, B. K. Kobilka, J Biol Chem 267, 3530-8. (1992).

36. R. M. Gage, K. A. Kim, T. T. Cao, M. von Zastrow, J Biol Chem 276, 44712-20. (2001).

37. U. E. Petaja-Repo et al., J Biol Chem 276, 4416-23. (2001).

38. K. Chaturvedi, P. Bandari, N. Chinen, R. D. Howells, J Biol Chem 276, 12345-55. (2001).

39. S. K. Shenoy, P. H. McDonald, T. A. Kohout, R. J. Lefkowitz, Science 294, 1307-13. (2001).

40. M. Cong et al., J Biol Chem 276, 45145-52. (2001).

41. P. Tsao, M. von Zastrow, Curr Opin Neurobiol 10, 365-9. (2000).

42. T. L. Yaksh, J Pharmacol Exp Ther 226, 303-16. (1983).

43. H. Wang, V. M. Pickel, J Neurosci 21, 3242-50. (2001).

44. These cells had a doubling time 3 times that of cells expressing endogenous levels of GASP1.

45. Keith et al., J Biol Chem 271, 19021 (1996).

46. The sequence of the DOR tail domain used as bait in the yeast two hybrid screen is as follows (in single-letter amino acid code):

| | |
|---|---|
| SLNPVLYAFLDENFKRCFRQLCRTPCGRQEPGSLRRPRQATTRERVTACTPSDGPGG | SEQ ID NO: 3 |
| GAA | |

### Example 2

### GASP2

The gene for GASP2 was identified using a BLAST (Basic Local Alignment Search Tool) query of the Human nucleotide database accessible through the National Center for Biotechnology Information (www.ncbi.nlm.nih.gov). Using both the nucleotide and amino acid sequence of GASP1 (also referred to as KIAA0443, Genbank accession #NM_{—}014710), the mRNA transcript for the putative GASP2 gene, “hypothetical protein BC013576” accession# NM_{—}138437.1, was discovered to have very high homology at both the nucleotide and amino acid levels.

Genomic Organization

Both GASP homologues closely reside on the X chromosome at the interval Xq22.1 in Homo sapiens and XE3 in Mus musculus (amino acid sequence Genbank accession# XM_{—}142154.1). Based on the X chromosome sequence, the GASP1 and GASP2 coding regions are separated by 38,794 base pairs in Mus and 56,772 base pairs in Homo, GASP2 being qter to GASP. A similar genomic structure is also conserved in other mammalian species, such as Bos and Rattus. The GASP family of genes appears to be the result of gene duplication, sharing very similar intron/exon structure, the coding regions residing in a single large exon. Smaller exons exist, making up the uncharacteristically long 5′ untranslated regions with putative Internal Ribosomal Entry sites (IRES) regulating translation initiation of both homologues. The introns of the 5′ untranslated regions are uncharacteristically small for most metazoans, averaging 100 to 150 base pairs.

GASP2 Expression

According to EST (expressed sequence tag) data from Genbank, GASP2 expression pattern is similar to that of GASP: hypothalamus, cerebellum, optic nerve, whole brain, spinal cord, prostate, uterus, retina and eyes, adrenal gland, kidney, testis, pituitary, and heart.

Human GASP1/GASP2 Amino Acid Alignment

44% identity overall

˜50% Identity in NH2-terminal 500 AA

63% identity in COOH-terminal 650 AA

| | | | |
|---|---|---|---|
| GASP: | 1 | MTGAEIESGAQVKPEKKPGEEVVGGAEIENDVPLVVRPKVRTQA---------------- | 44 |
| | | MTGAEIE AQ KPEKK GEEV+G E ENDVPLVVRPKVRTQA | |
| GASP2: | 1 | MTGAEIEPSAQAKPEKKAGEEVIAGPERENDVPLVVRPKVRTQATTGARPKTETKSVPAA | 60 |
| GASP: | 45 | ------------------QIMPGARPKNKSKVMPGASTKVETSAVGGARPKSKAKAIPVS | 86 |
| | | Q+M GARPK +++ + GA K + AVGGAR K+ AKAIP + | |
| GASP2: | 61 | RPKTEAQAMSGARPKTEVQVMGGARPKTEAQGITGARPKTDARAVGGARSKTDAKAIPGA | 120 |
| GASP: | 87 | RFKEEAQMWAQPRFGAERLSKTERNSQTNIIASPLVSTDSVLVAKTKYLSEDRELVNTDT | 146 |
| | | R K+EAQ WAQ FG E +S+ E SQTN +A PL + +S V K+K LS DRELVN D | |
| GASP2: | 121 | RPKDEAQAWAQSEFGTEAVSQAEGVSQTNAVAWPLATAESGSVTKSKGLSMDRELVNVDA | 180 |
| GASP: | 147 | ESFPRRKAHYQAGFQPSFRSKEETNMGSWCCPRPTSKQEASPNSDFKWVDK-SVSSLFWS | 205 |
| | | E+FP + Q G QP F EETNMGSWC RP +++EAS S F D+ S +S FW+ | |
| GASP2: | 181 | ETFPGTQG--QKGIQPWFGPGEETNMGSWCYSRPRAREEASNESGFWSADETSTASSFWT | 238 |
| GASP: | 206 | GDEVTAKFHPGNRVKDSNRSMHMANQEANTMSRSQTNQELYIASSSGSEDESVKTPWFWA | 265 |
| | | G+E + + P R + +RS H A + N SR ++ QE Y+ S SGSEDE+ FW | |
| GASP2: | 239 | GEETSVRSWP--REESNTRSRHRAKHQTNPRSRPRSKQEAYVDSWSGSEDEASNPFSFWV | 296 |
| GASP: | 266 | RDKTNTWSGPREDPNSRSRFRSKKEVYVESSSGSEHEDHLESWFGAGKEGKFRSKMRAGK | 325 |
| | | + TN PR + | |
| GASP2: | 297 | GENTNNLFRPR----------------------------------------------VRE | 310 |
| GASP: | 326 | EANNRARHRAKREACIDFMPGSIDVIKKESCFWPEENANTFSRPMIKKEARARAMTKEEA | 385 |
| | | EAN R++ R RE C F S D K+S P E AN+ R R KE + | |
| GASP2: | 311 | EANIRSKLRTNREDC--FESESEDEFYKQSWVLPGEEANS----------RFRHRDKEDP | 358 |
| GASP: | 386 | KTKARARAKQEARSEEEALIGTWFWATDESSMADEASIESSLQVEDESIIGSWFWTEEEA | 445 |
| | | T + RA+++ S+ | |
| GASP2: | 359 | NTALKLRAQKDVDSDRV------------------------------------------- | 375 |
| GASP: | 446 | SMGTGASSKSRPRTDGERIGDSLFGAREKTSMKTGAEATSESILAADDEQVIIGSWFWAG | 505 |
| | | K PR | |
| GASP2: | 376 | --------KQEPRF---------------------------------------------- | 381 |
| GASP: | 506 | EEVNQEAEEETIFGSWFWVIDAASVESGVGVSCESRTRSEEEEVIGPWFWSGEQVDIEAG | 565 |
| GASP2: | 381 | ------------------------------------------------------------ | 381 |
| GASP: | 566 | IGEEARPGAEEETIFGSWFWAENQTYMDCRAETSCDTMQGAEEEEPIIGSWFWTRVEACV | 625 |
| | | EEE IIGSWFW EA + | |
| GASP2: | 382 | ------------------------------------------EEEVIIGSWFWAEKEASL | 399 |
| GASP: | 626 | EGDVNSKSSLEDKEEAMIPCFGAKEEVSMKHGTGVRCRFMAGAEETNNKSCFWAEKEPCM | 685 |
| | | EG G C | |
| GASP2: | 400 | EG-----------------------------GASAIC----------------------- | 407 |
| GASP: | 686 | YPAGGGSWKSRPEEEEDIVNSWFWSRKYTKPEAIIGSWLWATEESNIDGTGEKAKLLTEE | 745 |
| GASP2: | 407 | ------------------------------------------------------------ | 407 |
| GASP: | 746 | ETIINSWFWKEDEAISEATDREESRPEAEEGDIVGSWFWAGEEDRLEPAAETREEDRLAA | 805 |
| | | ES P EEG I GS +WA E+ L | |
| GASP2: | 408 | ----------------------ESEPGTEEGAIGGSAYWAEEKSSL-------------- | 431 |
| GASP: | 806 | EKEGIVGSWFGAREETIRREAGSCSKSSPKAEEEEVIIGSWFWEEEASPEAVAGVGFESK | 865 |
| GASP2: | 431 | ------------------------------------------------------------ | 431 |
| GASP: | 866 | PGTEEEEITVGSWFWPEEEASIQAGSQAVEEMESETEEETIFGSWFWDGKEVSEEAGPCC | 925 |
| | | G+ A EE + E+EEE IFGSWFWD E + PC | |
| GASP2: | 432 | ------------------------GAVAREEAKPESEEEAIFGSWFWDRDEACFDLNPCP | 467 |
| GASP: | 926 | VSKPEDDEEMIVESWFWSRDKAIKETGTVATCESKPENEEGAIVGSWFEAEDEVDNRTDN | 985 |
| | | V K D RD A | |
| GASP2: | 468 | VYKVSDR----------FRDAA-------------------------------------- | 479 |
| GASP: | 986 | GSNCGSRTLADEDEAIVGSWFWAGDEAHFESNPSPVFRAICRSTCSVEQEPDPSRRPQSW | 1045 |
| | | +E + S RPQ+W | |
| GASP2: | 480 | ------------------------------------------------EELNASSRPQTW | 491 |
| GASP: | 1046 | EEVTVQFKPGPWGRVGFPSISPFRFPKEAASLFCEMFGGKPRNMVLSPEGEDQESLLQPD | 1105 |
| | | +EVTV+FKPG + VGF S SPF P+EA+ EM KP+N+ LSPEGE+QESLLQPD | |
| GASP2: | 492 | DEVTVEFKPGLFHGVGFRSTSPFGIPEEAS----EMLEAKPKNLELSPEGEEQESLLQPD | 547 |
| GASP: | 1106 | QPSPEFPFQYDPSYRSVQEIREHLRAKESTEPESSSCNCIQCELKIGSEEFEELLLLMEK | 1165 |
| | | QPSPEF FQYDPSYRSV+EIREHLRA+ES E ES SC+CIQCELKIGSEEFEE LLLM+K | |
| GASP2: | 548 | QPSPEFTFQYDPSYRSVREIREHLRARESAESESWSCSCIQCELKIGSEEFEEFLLLMDK | 607 |
| GASP: 1166 | IRDPFIHEISKIAMGMRSASQFTRDFIRDSGVVSLIETLLNYPSSRVRTSFLENMIRMAP | 1225 | |
| | | IRDPFIHEISKIAMGMRSASQFTRDFIRDSGVVSLIETLLNYPSSRVRTSFLENMI MAP | |
| GASP2: | 608 | IRDPFIHEISKIAMGMRSASQFTRDFIRDSGVVSLIETLLNYPSSRVRTSFLENMIHMAP | 667 |
| GASP: | 1226 | PYPNLNIIQTYICKVCEETLAYSVDSPEQLSGIRMIRHLTTTTDYHTLVANYMSGFLSLL | 1285 |
| | | PYPNLN+I+T+IC+VCEETLA+SVDS EQL+GIRM+RHLT T DYHTL+ANYMSGFLSLL | |
| GASP2: | 668 | PYPNLNMIETFICQVCEETLAHSVDSLEQLTGIRMLRHLTMTIDYHTLIANYMSGFLSLL | 727 |
| GASP: | 1286 | ATGNAKTRFHVLKMLLNLSENLFMTKELLSAEAVSEFIGLFNREETNDNIQIVLAIFENI | 1345 |
| | | T NA+T+FHVLKMLLNLSEN + K+L SA+A+S F+GLFN EETNDNIQIV+ +F+NI | |
| GASP2: | 728 | TTANARTKFHVLKMLLNLSENPAVAKKLFSAKALSIFVGLFNIEETNDNIQIVIKMFQNI | 787 |
| GASP: | 1346 | GNNIKK-ETVFSDDDFNIEPLISAFHKVEKFAKELQGKTDNQNDPEGDQEN | 1395 |
| | | N IK + DDDF++EPLISAF + E+ AK+LQ + DNQNDPE Q++ | |
| GASP2: | 788 | SNIIKSGKMSLIDDDFSLEPLISAFREFEELAKQLQAQIDNQNDPEVGQQS | 838 |

What is claimed is:

1. A method of inhibiting agonist-induced down-regulation of a G protein-coupled receptor, the method comprising contacting cells comprising the G protein-coupled receptor with an effective amount of an inhibitor, wherein:
the G protein-coupled receptor is one that specifically binds to a polypeptide having the amino acid sequence of GASP SEQ ID NO:2 (GASP1) or GASP SEQ ID NO:6 (GASP2);
the inhibitor reduces specific binding of the G protein-coupled receptor to said polypeptide; and
an effective amount is an amount sufficient to reduce agonist-induced down-regulation of the G protein-coupled receptor in the cells.

2. The method of claim 1, wherein the inhibitor comprises a polypeptide that reduces agonist-induced down-regulation of the G protein-coupled receptor and comprises an amino acid sequence that has at least about 70% identity to GASP SEQ ID NO:2 (GASP1) or GASP SEQ ID NO:6 (GASP2) over a comparison window of at least 15 contiguous amino acids.

3. The method of claim 2, wherein the amino acid sequence has at least about 95% identity to GASP SEQ ID NO:2 (GASP1) or GASP SEQ ID NO:6 (GASP2) over a comparison window of at least 15 contiguous amino acids.

4. The method of claim 3, wherein the amino acid sequence comprises an amino acid subsequence of at least about 500 amino acids, of GASP SEQ ID NO:2 (GASP1) or GASP SEQ ID NO:6 (GASP2).

5. The method of claim 2, wherein identity is determined by a sequence alignment performed using BLASTP with default parameters set to measure 70% identity.

6. The method of claim 2, wherein the amino acid sequence defines a peptide that specifically binds to a G protein-coupled receptor.

7. The method of claim 6, wherein the peptide comprises a subsequence of at least about 500 amino acids of GASP SEQ ID NO:2 (GASP1) or GASP SEQ ID NO:6 (GASP2).

8. The method of claim 6, wherein the peptide reduces agonist-induced down-regulation of the G protein-coupled receptor.

9. The method of claim 8, wherein the peptide reduces agonist-induced down-regulation by at least about 20%, as determined by a radioligand binding assay.

10. The method of claim 2, wherein said contacting comprises administering a composition comprising the polypeptide to the cells.

11. The method of claim 2, wherein said contacting comprises administering a composition comprising a polynucleotide encoding the polypeptide to the cells, whereby said administration results in the expression of the polypeptide.

12. The method of claim 1, wherein the cells are in vitro.

13. The method of claim 1, wherein the cells are in vivo.

14. The method of claim 1, wherein the G protein-coupled receptor is selected from the group comprising the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

15. The method of claim 14, wherein G protein-coupled receptor is selected from the group comprising the delta opioid receptor, the kappa opioid receptor, the D2 dopamine receptor, the D4 dopamine receptor, the NK1 (substance P) receptor, the bradykinin B1 receptor, and US28.

16. The method of claim 15, wherein said contacting is performed by administering a composition comprising the inhibitor to a subject in need of pain reduction.

17. The method of claim 1, additionally comprising contacting the cells with an agonist of the G protein-coupled receptor in an amount sufficient to stimulate the G protein-coupled receptor.

18. A method of enhancing agonist-induced down-regulation of a G protein-coupled receptor, the method comprising contacting cells comprising the G protein-coupled receptor with an effective amount of a polypeptide that increases agonist-induced down-regulation of the G protein-coupled receptor, wherein:
the G protein-coupled receptor is one that specifically binds to a polypeptide having the amino acid sequence of GASP SEQ ID NO:2 (GASP1) or GASP SEQ ID NO:6 (GASP2);
the polypeptide comprises an amino acid sequence that has at least about 70% identity to GASP SEQ ID NO:2 (GASP1) or GASP SEQ ID NO:6 (GASP2) over a comparison window of at least 15 contiguous amino acids; and
an effective amount is an amount sufficient to increase agonist-induced down-regulation of the G protein-coupled receptor in the cells.

19. The method of claim 18, wherein the amino acid sequence has at least about 95% identity to GASP SEQ ID NO:2 (GASP1) or GASP SEQ ID NO:6 (GASP2) over a comparison window of at least 15 contiguous amino acids.

20. The method of claim 18, wherein identity is determined by a sequence alignment performed using BLASTP with default parameters set to measure 70% identity.

21. The method of claim 18, wherein the amino acid sequence defines a polypeptide or peptide that specifically binds to a G protein-coupled receptor.

22. The method of claim 21, wherein the polypeptide or peptide increases agonist-induced down-regulation of the G protein-coupled receptor.

23. The method of claim 22, wherein the peptide increases agonist-induced down-regulation by at least about 20%, as determined by a radioligand binding assay.

24. The method of claim 18, wherein said contacting comprises administering a composition comprising the polypeptide to the cells.

25. The method of claim 18, wherein said contacting comprises administering a composition comprising a polynucleotide encoding the polypeptide to the cells, whereby said administration results in the expression of the polypeptide.

26. The method of claim 18, wherein the cells are in vitro.

27. The method of claim 18, wherein the cells are in vivo.

28. The method of claim 18, wherein the wherein the G protein-coupled receptor is selected from the group comprising the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

29. The method of claim 18, additionally comprising contacting the cells with an agonist of the G protein-coupled receptor in an amount sufficient to stimulate the G protein-coupled receptor.

30. An isolated polypeptide comprising an amino acid sequence, wherein the amino acid sequence has at least about 70% identity to GASP SEQ ID NO:2 (GASP1) or GASP SEQ ID NO:6 (GASP2) over a comparison window of at least 15 contiguous amino acids.

31. The polypeptide of claim 30, wherein the amino acid sequence has at least about 95% identity to GASP SEQ ID NO:2 (GASP1) or GASP SEQ ID NO:6 (GASP2) over a comparison window of at least 15 contiguous amino acids.

32. The polypeptide of claim 30, wherein the amino acid sequence comprises an amino acid sequence, or subsequence of at least about 500 amino acids, of GASP SEQ ID NO:2 (GASP1) or GASP SEQ ID NO:6 (GASP2).

33. The polypeptide of claim 30, wherein identity is determined by a sequence alignment performed using BLASTP with default parameters set to measure 70% identity.

34. The polypeptide of claim 30, wherein the amino acid sequence defines a polypeptide or a peptide that specifically binds to a G protein-coupled receptor.

35. The polypeptide of claim 34, wherein the G protein-coupled receptor is selected from the group comprising the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

36. The polypeptide of claim 35, wherein the G protein-coupled receptor is the delta opioid receptor.

37. The polypeptide of claim 34, wherein the amino acid sequence defines a peptide that specifically binds to a G protein-coupled receptor.

38. The polypeptide of claim 37, wherein the peptide comprises a subsequence of at least about 500 amino acids of GASP SEQ ID NO:2 (GASP1) or GASP SEQ ID NO:6 (GASP2).

39. The polypeptide of claim 37, wherein the peptide reduces agonist-induced down-regulation of the G protein-coupled receptor.

40. The polypeptide of claim 38, wherein the peptide reduces agonist-induced down-regulation by at least about 20%, as determined by a radioligand binding assay.

41. The polypeptide of claim 30, wherein the polypeptide comprises a signal sequence.

42. The polypeptide of claim 30, wherein the polypeptide comprises an epitope tag.

43. A polypeptide comprising an amino acid sequence, wherein the amino acid sequence has at least about 70% identity to delta opioid receptor SEQ ID NO:3 over a comparison window of at least 15 contiguous amino acids and defines a peptide that specifically binds GASP SEQ ID NO:2 (GASP1) or GASP SEQ ID NO:6 (GASP2), wherein the polypeptide does not comprise more than about 50 contiguous amino acids of delta opioid receptor.

44. The polypeptide of claim 43, wherein the amino acid sequence has at least about 95% identity to delta opioid receptor SEQ ID NO:3 over a comparison window of at least 15 contiguous amino acids.

45. The polypeptide of claim 44, wherein the amino acid sequence comprises an amino acid sequence, or subsequence of at least about 30 amino acids, of delta opioid receptor SEQ ID NO:3.

46. The polypeptide of claim 43, wherein identity is determined by a sequence alignment performed using BLASTP with default parameters set to measure 70% identity.

47. An isolated polynucleotide that encodes the polypeptide of claim 30, 34, 37, 39, or 43

48. A vector that comprises that polynucleotide of claim 47.

49. The vector of claim 48, wherein the vector is an expression vector.

50. A host cell comprising the vector of claim 48.

51. A host cell comprising the vector of claim 49.

52. A method of producing a polypeptide comprising:
(a) culturing the host cell of claim 51 under conditions suitable for expression of the polypeptide; and
(b) recovering the expressed polypeptide from the culture.

53. A composition comprising the polypeptide of claim 30, 34, 37, 39, or 43 or a polynucleotide encoding said polypeptide, and a pharmaceutically acceptable carrier.

54. The composition of claim 53, wherein the composition comprises the polypeptide of claim 39 in an amount sufficient to inhibit agonist-induced down-regulation of the G protein-coupled receptor when the composition is contacted with cells comprising the G protein-coupled receptor.

55. The composition of claim 54, additionally comprising an agonist of the G protein-coupled receptor.

56. An antibody or antiserum that specifically binds to the polypeptide of claim 30, 34, 37, 39, or 43.

57. The antibody or antiserum of claim 56, wherein the antibody or antiserum is produced by:
(a) administering said polypeptide to a mammal; and
(b) recovering the antibody or antiserum from the mammal.

58. A method of prescreening for an agent that modulates agonist-induced down-regulation of a G protein-coupled receptor, the method comprising:
a) contacting a test agent with a polypeptide that specifically binds said G protein-coupled receptor and modulates agonist-induced down-regulation of the G protein-coupled receptor, wherein the polypeptide comprises an amino acid sequence that has at least about 70% identity to GASP SEQ ID NO:2 (GASP1) or GASP SEQ ID NO:6 (GASP2) over a comparison window of at least 15 contiguous amino acids or with a polynucleotide encoding said polypeptide; and
b) detecting specific binding of the test agent to the polypeptide or polynucleotide.

59. The prescreening method of claim 58, wherein the test agent is contacted with the polypeptide in the presence of the G protein-coupled receptor or a fragment thereof that is capable of specifically binding to the polypeptide.

60. The prescreening method of claim 58, wherein said method additionally comprises recording any test agent that specifically binds to the polypeptide or polynucleotide in a database of candidate agents that may inhibit agonist-induced down-regulation of the G protein-coupled receptor.

61. The prescreening method of claim 58, wherein said detecting comprises detecting specific binding of the test agent to the polypeptide.

62. The prescreening method of claim 58, wherein said detecting comprises detecting specific binding of the test agent to the polynucleotide.

63. The prescreening method of claim 58, wherein said contacting is in vitro.

64. A method of prescreening for an agent that inhibits or enhances agonist-induced down-regulation of a G protein-coupled receptor, the method comprising:
a) contacting a test agent with a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2); and
b) detecting specific binding of the test agent to the receptor or receptor fragment.

65. The prescreening method of claim 64, wherein said method additionally comprises recording any test agent that specifically binds to the G protein-coupled receptor or receptor fragment in a database of candidate agents that may inhibit or enhance agonist-induced down-regulation of the G protein-coupled receptor.

66. The prescreening method of claim 64, wherein the G protein-coupled receptor is selected from the group comprising the delta opioid receptor, the kappa opioid receptor, the D2 dopamine receptor, the D4 dopamine receptor, the beta 2 adrenergic receptor, the NK1 (substance P) receptor, the bradykinin B1 receptor, and US28.

67. The prescreening method of claim 64, wherein said contacting is in vitro.

68. A method of screening for an agent that inhibits or enhances agonist-induced down-regulation of a G protein-coupled receptor, said method comprising:
a) contacting a test agent with a cell comprising:
(i) a G protein-coupled receptor, or fragment thereof, wherein the G protein-coupled receptor or receptor fragment specifically binds to a polypeptide having the amino acid sequence of GASP SEQ ID NO:2 (GASP1) or GASP SEQ ID NO:6 (GASP2); and
(ii) a GASP polypeptide comprising SEQ ID NO:2 (GASP1) or SEQ ID NO:6 (GASP2) or an allelic or species variant thereof;
b) determining the level of:
(i) GASP polypeptide;
(ii) GASP RNA; or
(iii) agonist-induced down-regulation of the G protein-coupled receptor.

69. The screening method of claim 68, wherein said method additionally comprises recording any test agent that induces a difference in said level in a database of agents that inhibit or enhance agonist-induced down-regulation of the G protein-coupled receptor.

70. The screening method of claim 68, wherein the level of GASP polypeptide is determined.

71. The screening method of claim 68, wherein the level of GASP RNA is determined.

72. The screening method of claim 68, wherein the level of agonist-induced down-regulation of the G protein-coupled receptor is determined.

73. The screening method of claim 68, the cell is in vitro.

74. The screening method of claim 68, additionally comprising selecting a test agent that reduces the level of:
(i) GASP polypeptide;
(ii) GASP RNA; or
(iii) agonist-induced down-regulation of the G protein-coupled receptor.
as an inhibitor of agonist-induced down-regulation of the G protein-coupled receptor.

75. The screening method of claim 68, wherein said determining comprises determining the level of agonist-induced down-regulation of the G protein-coupled receptor by a radioligand binding assay.

76. The screening method of claim 68, additionally comprising selecting a test agent that increases the level of:
(i) GASP polypeptide;
(ii) GASP RNA; or
(iii) agonist-induced down-regulation of the G protein-coupled receptor as an enhancer of agonist-induced down-regulation of the G protein-coupled receptor.

77. The screening method of claim 74 or 76, additionally comprising combining the inhibitor or enhancer, respectively, with a pharmaceutically acceptable carrier.

78. The screening method of claim 77, additionally comprising combining the inhibitor or enhancer with an agonist of the G protein-coupled receptor.
